(19) United States
(12) Patent Application Publication
(10) Pub. No.: US 2011/0045463 A1
(43) Pub. Date: Feb. 24, 2011

(54) METHOD FOR MEASURING DNA METHYLATION

(75) Inventors: Yoshitaka Tomigahara, Osaka (JP); Hirokazu Tarui, Osaka (JP)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Sumitomo Chemical Company, Limited, Chuo-ku, Tokyo (JP)

(21) Appl. No.: 12/521,236

(22) PCT Filed: Dec. 25, 2007
(86) PCT No.: PCT/JP2007/075361

§ 371 Date: Jul. 27, 2009

(30) Foreign Application Priority Data
Dec. 25, 2006 (JP) 2006-347300

(51) Int. Cl. C12Q 1/68

(52) U.S. Cl. 435/6

(57) ABSTRACT

The present invention relates to a method of measuring the content of methylated DNA in a DNA region of interest in a genomic DNA contained in a biological specimen, and so on.

### TECHNICAL FIELD

The present invention relates to a method of measuring the content of methylated DNA in a DNA region of interest in a genomic DNA contained in a biological specimen, and so on.

### BACKGROUND ART

As a method for evaluating the methylation state of DNA in an objective DNA region in a genomic DNA contained in a biological specimen, for example, there is known a method of measuring the content of methylated DNA in an objective DNA region in a genomic DNA (see, for example, Nucleic Acids Res., 1994, Aug. 11; 22(15): 2990-7, and Proc. Natl. Acad. Sci. U.S.A., 1997, Mar. 18; 94(6): 2284-9 for reference). In such a measuring method, first, it is necessary to extract DNA containing the objective DNA region from a DNA sample derived from a genomic DNA, and the extracting operation is complicated.

As a method of measuring the content of methylated DNA in an objective region of extracted DNA, for example, (1) a method of amplifying an objective region by subjecting the DNA to a chain reaction for DNA synthesis by DNA polymerase after modification of the DNA with a sulfite or the like (Polymerase Chain Reaction; hereinafter also referred to as PCR), and (2) a method of amplifying an objective region by subjecting the DNA to PCR after digestion of the DNA using a methylation sensitive restriction enzyme are known. Both of these methods require time and labor for DNA modification for detection of methylation, subsequent purification of the product, preparation of a reaction system for PCR, and checking of DNA amplification.

### DISCLOSURE OF THE INVENTION

It is an object of the present invention to provide a method of measuring the content of methylated DNA in an objective DNA region in a genomic DNA contained in a biological specimen in a simple and convenient manner.

That is, the present invention provides:

1. a method of measuring the content of methylated DNA in an objective DNA region in a genomic DNA contained in a biological specimen, comprising:
(1) First step of causing base-pairing between a single-stranded DNA (plus strand) containing an objective DNA region and a single-stranded immobilized oligonucleotide having a nucleotide sequence that is complementary to the objective DNA region of the single-stranded DNA, and selecting the single-stranded DNA from a DNA sample derived from a genomic DNA contained in a biological specimen, and forming double-stranded DNA made up of the selected single-stranded DNA and the single-stranded immobilized oligonucleotide that are base-paired;
(2) Second step of digesting the double-stranded DNA formed in First step with one or more kind of methylation-sensitive restriction enzyme, and removing generated free digests (double-stranded DNA containing one or more unmethylated CpG pair in a recognition site of the methylation-sensitive restriction enzyme); and
(3) Third step comprising, as pre steps of each of the following regular steps:

Step (First pre step) of temporarily separating the formed double-stranded DNA (formed double-stranded DNA not containing an unmethylated CpG pair in a recognition site of the methylation-sensitive restriction enzyme) which is an undigested substance obtained in Second step, in a single-stranded state;

Step (Second pre step) comprising:

Step (Second (A) pre step) of causing base-pairing between the generated free single-stranded DNA (plus strand) and the single-stranded immobilized oligonucleotide, and selecting the generated free single-stranded DNA, and forming double-stranded DNA made up of the selected single-stranded DNA and the single-stranded immobilized oligonucleotide that are base-paired, and

Step (Second (B) pre step) of making the double-stranded DNA formed in Step (Second (A) pre step) into double-stranded DNA extended and formed from the selected single-stranded DNA by extending a primer once using the selected single-stranded DNA as a template and the single-stranded immobilized oligonucleotide as a primer; and

Step (Third pre step) of temporarily separating the double-stranded DNA (extensionally-formed double-stranded DNA not containing an unmethylated CpG pair in a recognition site of the methylation-sensitive restriction enzyme) extended and formed in Second pre step into a single-stranded DNA (plus strand) and a single-stranded DNA (minus strand), and as regular steps,

(a) Step A (a regular step) comprising:

Step A1 of causing base-paring between the generated single-stranded DNA (plus strand) and the single-stranded immobilized oligonucleotide (minus strand), and selecting the single-stranded DNA, and

Step A2 of extending and forming double-stranded DNA from the single-stranded DNA by one extension of a primer using the single-stranded DNA selected in Step A1 as a template and the single-stranded immobilized oligonucleotide as a primer,

(b) Step B (a regular step) of making the single-stranded DNA into extensionally-formed double-stranded DNA by one extension of oligonucleotide by using the single-stranded generated DNA (minus strand) as a template, and oligonucleotide (a reverse primer) having a nucleotide sequence (plus strand) complementary to a partial nucleotide sequence (minus strand) of a nucleotide sequence possessed by the DNA in a single-stranded state (minus strand), wherein the partial nucleotide sequence (minus strand) is positioned on the 3′-end side than the 3′-end of the nucleotide sequence (minus strand) complementary to the nucleotide sequence (plus strand) of the objective DNA region, and is not usable in an extension reaction using the single-stranded immobilized oligonucleotide as a template, as an extension primer,

wherein the methylated DNA in the objective DNA region is amplified to a detectable level by repeating each regular step of Third step after temporarily separating the extensionally-formed double-stranded DNA obtained in each regular step into a single-stranded state, and the amplified DNA is quantified (hereinafter, also referred to as the present measuring method);

2. the method according to the above item 1., wherein in First step, base pairing is conducted in a reaction system containing a divalent cation when the single-stranded DNA (plus strand) containing the objective DNA region and the single-stranded immobilized oligonucleotide having a nucleotide sequence complementary to the objective DNA region of the single-stranded DNA are base-paired;
3. the method according to the above item 2., wherein the divalent cation is a magnesium ion;
4. the method according to any one of the items 1. to 3., further comprising in a previous operation stage of First pre step in Third step:

a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and

additionally comprising the following one step as a respective regular step of Third step as described in any of the items 1. to 3.:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer;

5. the method according to any one of the items 1. to 3., further comprising in a post operation stage of First pre step in Third step:

a step (Additional pre step) of adding single-stranded oligonucleotide (minus strand) in a free state having a nucleotide sequence complementary to a part of the 3′-end of single-stranded DNA (plus strand) containing the objective DNA region into a reaction system, and

a step (additional re-pre step) of temporarily separating into a single-stranded state double-stranded DNA (extensionally-formed double-stranded DNA not containing an unmethylated CpG pair in the recognition site of the methylation-sensitive restriction enzyme) which is an undigested substance obtained through Second step and Additional pre step, and

additionally comprising the following one step as a respective regular step of Third step as described in any of the items 1. to 3.:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer (hereinafter, also referred to as the present methylation rate measuring method);

6. the method according to any one of the items 1. to 3., further comprising in a previous operation stage of Third pre step in Third step:

a step (Additional pre step) of adding single-stranded oligonucleotide (minus strand) in a free state having a nucleotide sequence complementary to a part of the 3′-end of single-stranded DNA (plus strand) containing the objective DNA region into a reaction system, and

additionally comprising the following one step as a respective regular step of Third step as described in any of the items 1. to 3.:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer;

7. the method according to any one of the items 1. to 3., further comprising in a post operation stage of Third pre step in Third step:

a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and

a step (additional re-pre step) of temporarily separating into a single-stranded state double-stranded DNA (extensionally-formed double-stranded DNA not containing an unmethylated CpG pair in the recognition site of the methylation sensitive restriction enzyme) which is an undigested substance obtained through Second step and Additional pre step, and

additionally comprising the following one step as a respective regular step of Third step as described in any of the items 1. to 3.:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer;

8. a method of measuring a methylation rate additionally comprising the following two steps as steps of the method as described in any one of the items 1. to 7.:
(4) Fourth step of amplifying DNA (total amount of methylated DNA and unmethylated DNA) of the objective DNA region to a detectable level by conducting Third step in the method as described in any one of the items 1. to 7. after conducting First step in the method as described in any one of the items 1. to 7. without conducting Second step in the method as described in any one of the items 1. to 7., and quantifying the amplified DNA; and
(5) Fifth step of calculating a rate of methylated DNA in the objective DNA region based on a difference obtained by comparing the DNA amount quantified by Third step as described in any one of the items 1. to 7., and the DNA amount quantified in Fourth step;
9. the method as described in any one of the items 1. to 8., wherein the biological specimen is mammalian serum or plasma;
10. the method as described in any one of the items 1. to 8., wherein the biological specimen is mammalian blood or bodily secretion;
11. the method as described in any one of the items 1. to 8., wherein the biological specimen is a cell lysate or a tissue lysate;
12. the method as described in any one of the items 1. to 11., wherein the DNA sample derived from a genomic DNA contained in a biological specimen is a DNA sample digested in advance with a restriction enzyme whose recognition cleavage site excludes the objective DNA region possessed by the a genomic DNA;
13. the method as described in any one of the items 1. to 12., wherein the DNA sample derived from a genomic DNA contained in a biological specimen is a DNA sample digested with one or more kind of methylation-sensitive restriction enzyme;
14. the method as described in any one of the items 1. to 13., wherein the DNA sample derived from a genomic DNA contained in a biological specimen is a DNA sample purified in advance;
15. the method as described in any one of the items 1. to 12., wherein the one or more kind of methylation-sensitive restriction enzyme is a restriction enzyme having its recognition cleavage site in the objective DNA region possessed by a genomic DNA contained in the biological specimen;
16. the method as described in any one of the items 1. to 14., wherein the one or more kind of methylation sensitive restriction enzyme is HpaII or HhaI which is a methylation sensitive restriction enzyme; and so on.

### BRIEF DESCRIPTION OF THE DRAWINGS

FIG. 1 shows results of 1.5% agarose gel electrophoresis of amplified products which are obtained by conducting a treatment of either “A (untreated)” or “B (co-treated with HpaII and HhaI)” on the sample prepared in Example 1, and amplifying methylated DNA in the region having the nucleotide sequence of SEQ ID NO: 22 by PCR. In the drawing, results in a sample subjected to “A” treatment of a DNA fragment X2, a sample subjected to “B” treatment of a DNA fragment X2, a sample subjected to “A” treatment of a DNA fragment Y2, and a sample subjected to “B” treatment of a DNA fragment Y2 are shown from the leftmost lane.

FIG. 2 shows results of 1.5% agarose gel electrophoresis of amplified products which are obtained by conducting a treatment of either “A (untreated)” or “B (co-treated with HpaII and HhaI)” on the sample prepared in Example 2, and amplifying methylated DNA in the region having the nucleotide sequence of SEQ ID NO: 22 by PCR. In the drawing, results in a DNA marker “M”, Sample 1 subjected to “A” treatment of the DNA fragment Y2, Sample 2 subjected to “A” treatment of the DNA fragment Y2, Sample 3 subjected to “A” treatment of the DNA fragment Y2, Sample 1 subjected to “B” treatment of the DNA fragment Y2, Sample 2 subjected to “B” treatment of the DNA fragment Y2, Sample 3 subjected to “B” treatment of the DNA fragment Y2, Sample 1 subjected to “A” treatment of the DNA fragment X2, Sample 2 subjected to “A” treatment of the DNA fragment X2, Sample 3 subjected to “A” treatment of the DNA fragment X2, Sample 1 subjected to “B” treatment of the DNA fragment X2, Sample 2 subjected to “B” treatment of the DNA fragment X2, and Sample 3 subjected to “B” treatment of the DNA fragment X2 are shown from the leftmost lane.

FIG. 3 shows results of 1.5% agarose gel electrophoresis of amplified products which are obtained by conducting a treatment of either “A (untreated)” or “B (co-treated with HpaII and HhaI)” on a sample of a prepared DNA fragment X2 in Example 3, and amplifying methylated DNA in the region having the nucleotide sequence of SEQ ID NO: 22 by PCR. The leftmost lane in the drawing shows a DNA marker “M”. For Groups A and B, results in Sample “1” of a 10 pg DNA fragment X2/mL TE buffer solution, Sample “2” of a 1 pg DNA fragment X2/mL TE buffer solution, Sample “3” of a 0.1 pg DNA fragment X2/mL TE buffer solution, and Sample “4” of a 0 pg DNA fragment X2/mL TE buffer solution are respectively shown from left-hand.

FIG. 4 shows results of 1.5% agarose gel electrophoresis of amplified products which are obtained by conducting a treatment of either “A (untreated)” or “B (co-treated with HpaII and HhaI)” on a sample of a prepared DNA fragment Y2 in Example 3, and amplifying methylated DNA in the region having the nucleotide sequence of SEQ ID NO: 22 by PCR. The leftmost lane in the drawing shows a DNA marker “M”. For Groups A and B, results in Sample “1” of a 10 pg DNA fragment Y2/mL TE buffer solution, Sample “2” of a 1 pg DNA fragment Y2/mL TE buffer solution, Sample “3” of a 0.1 pg DNA fragment Y2/mL TE buffer solution, and Sample “4” of a 0 pg DNA fragment Y2/mL TE buffer solution are respectively shown from left-hand.

FIG. 5 shows results of 1.5% agarose gel electrophoresis of amplified products which are obtained by conducting a treatment of either “A (untreated)” or “B (co-treated with HpaII and HhaI)” on a sample of a prepared DNA fragment X2 in Example 4, and amplifying methylated DNA in the region having the nucleotide sequence of SEQ ID NO: 22 by PCR. The leftmost lane in the drawing shows a DNA marker “M”. For Groups A and B, results in Sample “1” of a 10 pg DNA fragment X2/mL rat serum solution, Sample “2” of a 1 pg DNA fragment X2/mL rat serum solution, Sample “3” of a 0.1 pg DNA fragment X2/mL rat serum solution, and Sample “4” of a 0 pg DNA fragment X2/mL rat serum solution are respectively shown from left-hand.

FIG. 6 shows results of 1.5% agarose gel electrophoresis of amplified products which are obtained by conducting a treatment of either “A (untreated)” or “B (co-treated with HpaII and HhaI)” on a sample of a prepared DNA fragment Y2 in Example 4, and amplifying methylated DNA in the region having the nucleotide sequence of SEQ ID NO: 22 by PCR. The leftmost lane in the drawing shows a DNA marker “M”. For Groups A and B, results in Sample “1” of a 10 pg DNA fragment Y2/mL rat serum solution, Sample “2” of a 1 pg DNA fragment Y2/mL rat serum solution, Sample “3” of a 0.1 pg DNA fragment Y2/mL rat serum solution, and Sample “4” of a 0 pg DNA fragment Y2/mL rat serum solution are respectively shown from left-hand.

FIG. 7 shows results of the amount of methylated DNA in a region having the nucleotide sequence of SEQ ID NO: 17 measured by real-time PCR, for the sample “(I)” prepared in Example 5, subjected to either “A (untreated)”, “B (treated with HpaII)”, “C (treated with HhaI)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C, and Group D are indicated.

FIG. 8 shows results of the amount of methylated DNA in a region having the nucleotide sequence of SEQ ID NO: 17 measured by real-time PCR, for the sample “(II)” prepared in Example 5, subjected to either “A (untreated)”, “B (treated with HpaII)”, “C (treated with HhaI)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C, and Group D are indicated.

FIG. 9 shows results of the amount of methylated DNA in a region having the nucleotide sequence of SEQ ID NO: 17 measured by real-time PCR, for the sample “(III)” prepared in Example 5, subjected to either “A (untreated)”, “B (treated with HpaII)”, “C (treated with HhaI)” or “D (co-treated with HpaII and HhaI)”.

The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C, and Group D are indicated.

FIG. 10 shows results of the amount of methylated DNA in a region having the nucleotide sequence of SEQ ID NO: 17 measured by real-time PCR, for the sample “(IV)” prepared in Example 5, subjected to either “A (untreated)”, “B (treated with HpaII)”, “C (treated with HhaI)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C, and Group D are indicated.

FIG. 11 shows results of the amount of methylated DNA in a region having the nucleotide sequence of SEQ ID NO: 17 measured by real-time PCR, for the sample “(V)” prepared in Example 5, subjected to either “A (untreated)”, “B (treated with HpaII)”, “C (treated with HhaI)” or “D (co-treated with HpaII and HhaI)”.

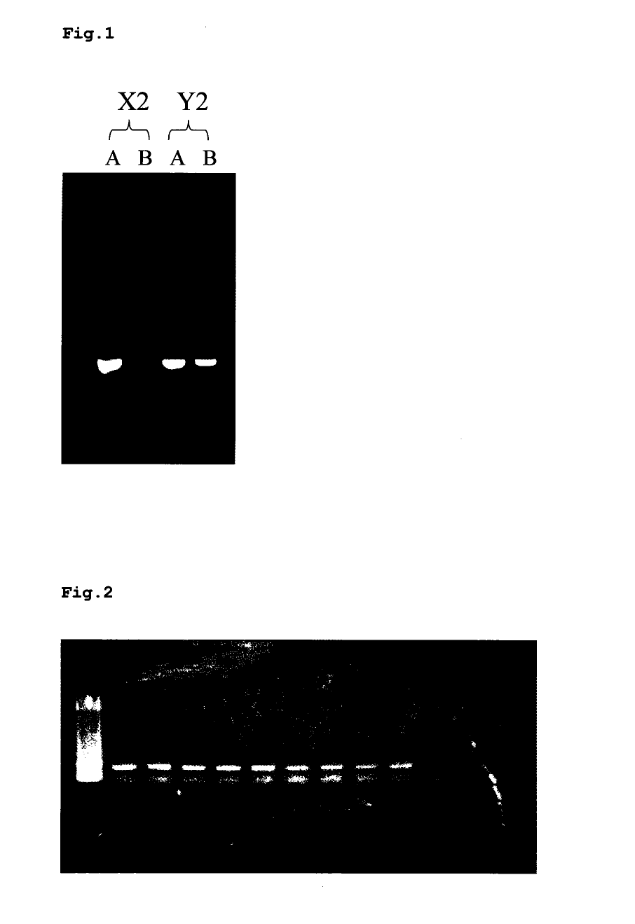

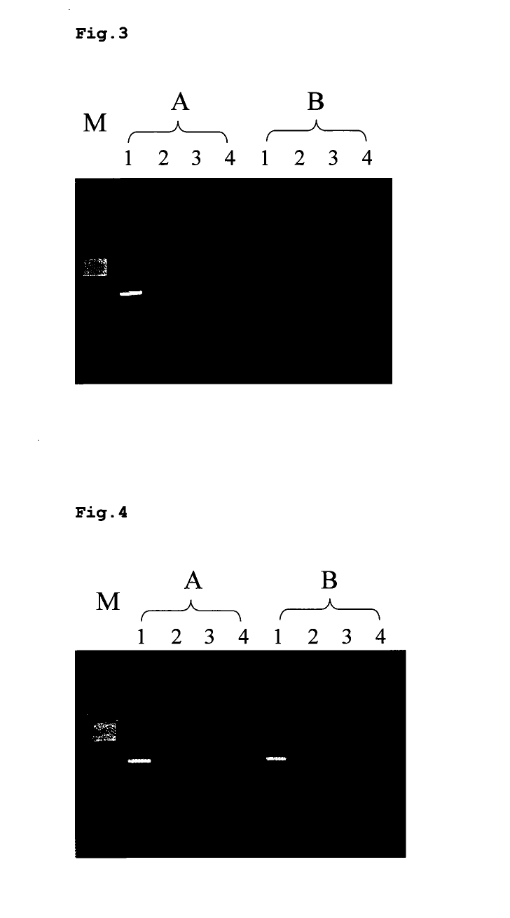

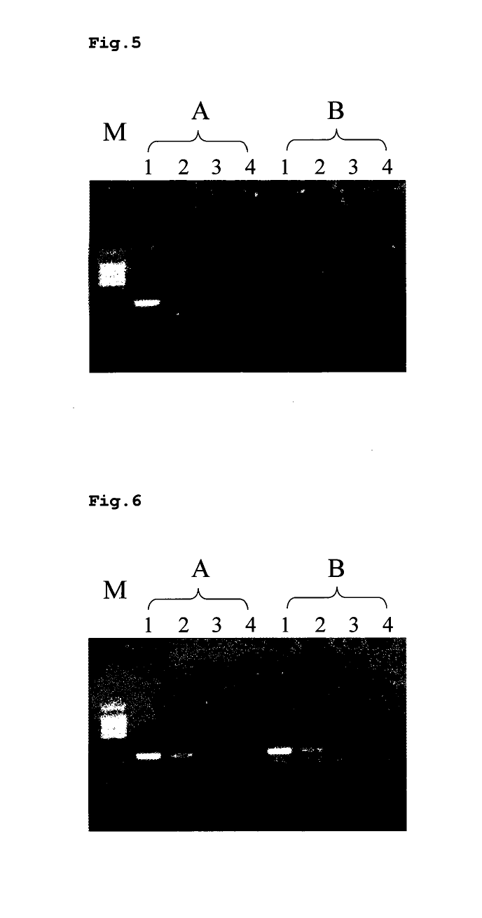

The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C, and Group D are indicated.

### BEST MODE FOR CARRYING OUT THE INVENTION

As the “biological specimen” in the present invention, for example, a cell lysate, a tissue lysate (here the term “tissue” is used in a broad sense including blood, lymph node and so on) or biological samples including bodily sections such as plasma, serum and lymph, bodily secretions (urine, milk and so on) and the like and a genomic DNA obtained by extracting these biological samples, in mammals can be recited. As a biological specimen, for example, samples derived from microorganisms, viruses and the like can be recited, and in such a case, “a genomic DNA” in the present measuring method also means genomic DNA of microorganisms, viruses and the like.

When the specimen derived from a mammal is blood, use of the present measuring method in a regular health check or a simple examination is expected.

For obtaining a genomic DNA from a specimen derived from a mammal, for example, DNA may be extracted using a commercially available DNA extraction kit.

When blood is used as a specimen, plasma or serum is prepared from blood in accordance with a commonly used method, and using the prepared plasma or serum as a specimen, free DNA (including DNA derived from cancer cells such as gastric cancer cells) contained in the specimen is analyzed. This enables analysis of DNA derived from cancer cells such as gastric cancer cells while avoiding DNA derived from hemocytes, and improves the sensitivity of detection of cancer cells such as gastric cancer cells and a tissue containing the same.

Usually, a gene (a genomic DNA) consists of four kinds of bases. In these bases, such a phenomenon is known that only cytosine is methylated, and such methylation modification of DNA is limited to cytosine in a nucleotide sequence represented by 5′-CG-3′ (C represents cytosine, and G represents guanine. Hereinafter, the nucleotide sequence is also referred to as “CpG”). The site to be methylated in cytosine is its position 5. In DNA replication prior to cell division, only cytosine in “CpG” of a template chain is methylated immediately after replication, however, cytosine in “CpG” of a newly-generated strand is immediately methylated by the action of methyltransferase. Therefore, the methylation state of DNA will be passed to new two sets of DNA even after DNA replication. The term “methylated DNA” in the present invention means DNA occurring by such methylation modification.

The term “CpG pair” in the present invention means double-stranded oligonucleotide in which a nucleotide sequence represented by CpG and a CpG that is complement with this are base-paired.

The term “objective DNA region” (hereinafter, also referred to as an “objective region”) used in the present invention means a DNA region for which presence or absence of methylation of cytosine included in the region is to be examined, and has a recognition site of at least one kind of methylation sensitive restriction enzyme. A DNA region containing at least one cytosine in a nucleotide sequence represented by CpG which is present in a nucleotide sequence of a promoter region, an untranslated region, or a translated region (coding region) of a useful protein gene such as Lysyl oxidase, HRAS-like suppressor, bA305P22.2.1, Gamma filamin, HAND1, Homologue of RIKEN 2210016F16, FLJ32130, PPARG angiopoietin-related protein, Thrombomodulin, p53-responsive gene 2, Fibrillin2, Neurofilament3, disintegrin and metalloproteinase domain 23, G protein-coupled receptor 7, G-protein coupled somatostatin and angiotensin-like peptide receptor, Solute carrier family 6 neurotransmitter transporter noradrenalin member 2 and so on can be recited.

To be more specific, when the useful protein gene is a Lysyl oxidase gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Lysyl oxidase gene derived from human, and a promoter region located 5′ upstream of the same can be recited, and more concretely, the nucleotide sequence of SEQ ID NO: 1 (corresponding to a nucleotide sequence represented by base No. 16001 to 18661 in the nucleotide sequence described in Genbank Accession No. AF270645) can be recited. In the nucleotide sequence of SEQ ID NO: 1, ATG codon encoding methionine at amino terminal of Lysyl oxidase protein derived from human is represented in base No. 2031 to 2033, and a nucleotide sequence of the above exon 1 is represented in base No. 1957 to 2661. Cytosine in the nucleotide sequence represented by CpG which is present in the nucleotide sequence of SEQ ID NO: 1, in particular, cytosine in CpG which is present in a region where CpGs are densely present in the nucleotide sequence of SEQ ID NO: 1 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1560, 1574, 1600, 1623, 1635, 1644, 1654, 1661, 1682, 1686, 1696, 1717, 1767, 1774, 1783, 1785, 1787, 1795 and so on in the nucleotide sequence of SEQ ID NO: 1 can be recited.

To be more specific, when the useful protein gene is a HRAS-like suppressor gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a HRAS-like suppressor gene derived from human, and a promoter region located 5′ upstream of the same can be recited, and more concretely, the nucleotide sequence of SEQ ID NO: 2 (corresponding to a nucleotide sequence represented by base No. 172001 to 173953 in the nucleotide sequence described in Genbank Accession No. AC068162) can be recited. In the nucleotide sequence of SEQ ID NO: 2, the nucleotide sequence of exon 1 of a HRAS-like suppressor gene derived from human is represented in base No. 1743 to 1953. Cytosine in the nucleotide sequence represented by CpG which is present in the nucleotide sequence of SEQ ID NO: 2, in particular, cytosine in CpG which is present in a region where CpGs are densely present in the nucleotide sequence of SEQ ID NO: 2 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1316, 1341, 1357, 1359, 1362, 1374, 1390, 1399, 1405, 1409, 1414, 1416, 1422, 1428, 1434, 1449, 1451, 1454, 1463, 1469, 1477, 1479, 1483, 1488, 1492, 1494, 1496, 1498, 1504, 1510, 1513, 1518, 1520 and so on in the nucleotide sequence of SEQ ID NO: 2 can be recited.

To be more specific, when the useful protein gene is a bA305P22.2.1 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a bA305P22.2.1 gene derived from human, and a promoter region located 5′ upstream of the same can be recited, and more concretely, the nucleotide sequence of SEQ ID NO: 3 (corresponding to a nucleotide sequence represented by base No. 13001 to 13889 in the nucleotide sequence described in Genbank Accession No. AL121673) can be recited. In the nucleotide sequence of SEQ ID NO: 3, ATG codon encoding methionine at amino terminal of bA305P22.2.1 protein derived from human is represented in base No. 849 to 851, and a nucleotide sequence of the above exon 1 is represented in base No. 663 to 889. Cytosine in the nucleotide sequence represented by CpG which is present in the nucleotide sequence of SEQ ID NO: 3, in particular, cytosine in CpG which is present in a region where CpGs are densely present in the nucleotide sequence of SEQ ID NO: 3 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 335, 337, 351, 363, 373, 405, 424, 427, 446, 465, 472, 486 and so on in the nucleotide sequence of SEQ ID NO: 3 can be recited.

To be more specific, when the useful protein gene is a Gamma filamin gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Gamma filamin gene derived from human, and a promoter region located 5′ upstream of the same can be recited, and more concretely, the nucleotide sequence of SEQ ID NO: 4 (corresponding to a complementary sequence to a nucleotide sequence represented by base No. 63528 to 64390 in the nucleotide sequence described in Genbank Accession No. AC074373) can be recited. In the nucleotide sequence of SEQ ID NO: 4, ATG codon encoding methionine at amino terminal of Gamma filamin protein derived from human is represented in base No. 572 to 574, and a nucleotide sequence of the above exon 1 is represented in base No. 463 to 863. Cytosine in the nucleotide sequence represented by CpG which is present in the nucleotide sequence of SEQ ID NO: 4, in particular, cytosine in CpG which is present in a region where CpGs are densely present in the nucleotide sequence of SEQ ID NO: 4 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 333, 337, 350, 353, 360, 363, 370, 379, 382, 384, 409, 414, 419, 426, 432, 434, 445, 449, 459, 472, 474, 486, 490, 503, 505 and so on in the nucleotide sequence of SEQ ID NO: 4 can be recited.

To be more specific, when the useful protein gene is a HAND1 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a HAND1 gene derived from human, and a promoter region located 5′ upstream of the same can be recited, and more concretely, the nucleotide sequence of SEQ ID NO: 5 (corresponding to a complementary sequence to a nucleotide sequence represented by base No. 24303 to 26500 in the nucleotide sequence described in Genbank Accession No. ACO26688) can be recited. In the nucleotide sequence of SEQ ID NO: 5, ATG codon encoding methionine at amino terminal of HAND1 protein derived from human is represented in base No. 1656 to 1658, and a nucleotide sequence of the above exon 1 is represented in base No. 1400 to 2198. Cytosine in the nucleotide sequence represented by CpG which is present in the nucleotide sequence of SEQ ID NO: 5, in particular, cytosine in CpG which is present in a region where CpGs are densely present in the nucleotide sequence of SEQ ID NO: 5 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1153, 1160, 1178, 1187, 1193, 1218, 1232, 1266, 1272, 1292, 1305, 1307, 1316, 1356, 1377, 1399, 1401, 1422, 1434 and so on in the nucleotide sequence of SEQ ID NO: 5 can be recited.

To be more specific, when the useful protein gene is a Homologue of RIKEN 2210016F16 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Homologue of RIKEN 2210016F16 gene derived from human, and a promoter region located 5′ upstream of the same can be recited, and more concretely, the nucleotide sequence of SEQ ID NO: 6 (corresponding to a complementary nucleotide sequence to a nucleotide sequence represented by base No. 157056 to 159000 in the nucleotide sequence described in Genbank Accession No. AL354733) can be recited. In the nucleotide sequence of SEQ ID NO: 6, a nucleotide sequence of exon 1 of a Homologue of a RIKEN 2210016F16 gene derived from human is represented in base No. 1392 to 1945. Cytosine in the nucleotide sequence represented by CpG which is present in the nucleotide sequence of SEQ ID NO: 6, in particular, cytosine in CpG which is present in a region where CpGs are densely present in the nucleotide sequence of SEQ ID NO: 6 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1172, 1175, 1180, 1183, 1189, 1204, 1209, 1267, 1271, 1278, 1281, 1313, 1319, 1332, 1334, 1338, 1346, 1352, 1358, 1366, 1378, 1392, 1402, 1433, 1436, 1438 and so on in the nucleotide sequence of SEQ ID NO: 6 can be recited.

To be more specific, when the useful protein gene is a FLJ32130 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a FLJ32130 gene derived from human, and a promoter region located 5′ upstream of the same can be recited, and more concretely, the nucleotide sequence of SEQ ID NO: 7 (corresponding to a complementary nucleotide sequence to a nucleotide sequence represented by base No. 1 to 2379 in the nucleotide sequence described in Genbank Accession No. AC002310) can be recited. In the nucleotide sequence of SEQ ID NO: 7, ATG codon encoding methionine at amino terminal of FLJ32130 protein derived from human is represented in base No. 2136 to 2138, and a nucleotide sequence assumed to be the above exon 1 is represented in base No. 2136 to 2379. Cytosine in the nucleotide sequence represented by CpG which is present in the nucleotide sequence of SEQ ID NO: 7, in particular, cytosine in CpG which is present in a region where CpGs are densely present in the nucleotide sequence of SEQ ID NO: 7 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1714, 1716, 1749, 1753, 1762, 1795, 1814, 1894, 1911, 1915, 1925, 1940, 1955, 1968 and so on in the nucleotide sequence of SEQ ID NO: 7 can be recited.

To be more specific, when the useful protein gene is a PPARG angiopoietin-related protein gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a PPARG angiopoietin-related protein gene derived from human, and a promoter region located 5′ upstream of the same can be recited, and more concretely, the nucleotide sequence of SEQ ID NO: 8 can be recited. In the nucleotide sequence of SEQ ID NO: 8, ATG codon encoding methionine at amino terminal of PPARG angiopoietin-related protein derived from human is represented in base No. 717 to 719, and a nucleotide sequence of the 5′ side part of the above exon 1 is represented in base No. 1957 to 2661. Cytosine in the nucleotide sequence represented by CpG which is present in the nucleotide sequence of SEQ ID NO: 8, in particular, cytosine in CpG which is present in a region where CpGs are densely present in the nucleotide sequence of SEQ ID NO: 8 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 35, 43, 51, 54, 75, 85, 107, 127, 129, 143, 184, 194, 223, 227, 236, 251, 258 and so on in the nucleotide sequence of SEQ ID NO: 8 can be recited.

To be more specific, when the useful protein gene is a Thrombomodulin gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Thrombomodulin gene derived from human, and a promoter region located 5′ upstream of the same can be recited, and more concretely, the nucleotide sequence of SEQ ID NO: 9 (corresponding to a nucleotide sequence represented by base No. 1 to 6096 in the nucleotide sequence described in Genbank Accession No. AF495471) can be recited. In the nucleotide sequence of SEQ ID NO: 9, ATG codon encoding methionine at amino terminal of Thrombomodulin protein derived from human is represented in base No. 2590 to 2592, and a nucleotide sequence of the above exon 1 is represented in base No. 2048 to 6096. Cytosine in the nucleotide sequence represented by CpG which is present in the nucleotide sequence of SEQ ID NO: 9, in particular, cytosine in CpG which is present in a region where CpGs are densely present in the nucleotide sequence of SEQ ID NO: 9 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1551, 1571, 1579, 1581, 1585, 1595, 1598, 1601, 1621, 1632, 1638, 1645, 1648, 1665, 1667, 1680, 1698, 1710, 1724, 1726, 1756 and so on in the nucleotide sequence of SEQ ID NO: 9 can be recited.

To be more specific, when the useful protein gene is a p53-responsive gene 2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a p53-responsive gene 2 gene derived from human, and a promoter region located 5′ upstream of the same can be recited, and more concretely, the nucleotide sequence of SEQ ID NO: 10 (corresponding to a complementary sequence to a nucleotide sequence represented by base No. 113501 to 116000 in the nucleotide sequence described in Genbank Accession No. AC009471) can be recited. In the nucleotide sequence of SEQ ID NO: 10, a nucleotide sequence of exon 1 of a p53-responsive gene 2 gene derived from human is represented in base No. 1558 to 1808. Cytosine in the nucleotide sequence represented by CpG which is present in the nucleotide sequence of SEQ ID NO: 10 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1282, 1284, 1301, 1308, 1315, 1319, 1349, 1351, 1357, 1361, 1365, 1378, 1383 and so on in the nucleotide sequence of SEQ ID NO: 10 can be recited.

To be more specific, when the useful protein gene is a Fibrillin2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Fibrillin2 gene derived from human, and a promoter region located 5′ upstream of the same can be recited, and more concretely, the nucleotide sequence of SEQ ID NO: 11 (corresponding to a complementary sequence to a nucleotide sequence represented by base No. 118801 to 121000 in the nucleotide sequence described in Genbank Accession No. AC113387) can be recited. In the nucleotide sequence of SEQ ID NO: 11, a nucleotide sequence of exon 1 of a Fibrillin2 gene derived from human is represented in base No. 1091 to 1345. Cytosine in the nucleotide sequence represented by CpG which is present in the nucleotide sequence of SEQ ID NO: 11 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 679, 687, 690, 699, 746, 773, 777, 783, 795, 799, 812, 823, 830, 834, 843 and so on in the nucleotide sequence of SEQ ID NO: 11 can be recited.

To be more specific, when the useful protein gene is a Neurofilament3 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Neurofilament3 gene derived from human, and a promoter region located 5′ upstream of the same can be recited, and more concretely, the nucleotide sequence of SEQ ID NO: 12 (corresponding to a complementary sequence to a nucleotide sequence represented by base No. 28001 to 30000 in the nucleotide sequence described in Genbank Accession No. AF106564) can be recited. In the nucleotide sequence of SEQ ID NO: 12, a nucleotide sequence of exon 1 of a Neurofilament3 gene derived from human is represented in base No. 614 to 1694. Cytosine in the nucleotide sequence represented by CpG which is present in the nucleotide sequence of SEQ ID NO: 12 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 428, 432, 443, 451, 471, 475, 482, 491, 499, 503, 506, 514, 519, 532, 541, 544, 546, 563, 566, 572, 580 and so on in the nucleotide sequence of SEQ ID NO: 12 can be recited.

To be more specific, when the useful protein gene is a disintegrin and metalloproteinase domain 23 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a disintegrin and metalloproteinase domain 23 gene derived from human, and a promoter region located 5′ upstream of the same can be recited, and more concretely, the nucleotide sequence of SEQ ID NO: 13 (corresponding to a nucleotide sequence represented by base No. 21001 to 23300 in the nucleotide sequence described in Genbank Accession No. AC009225) can be recited. In the nucleotide sequence of SEQ ID NO: 13, a nucleotide sequence of exon 1 of a disintegrin and metalloproteinase domain 23 gene derived from human is represented in base No. 1194 to 1630. Cytosine in the nucleotide sequence represented by CpG which is present in the nucleotide sequence of SEQ ID NO: 13 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 998, 1003, 1007, 1011, 1016, 1018, 1020, 1026, 1028, 1031, 1035, 1041, 1043, 1045, 1051, 1053, 1056, 1060, 1066, 1068, 1070, 1073, 1093, 1096, 1106, 1112, 1120, 1124, 1126 and so on in the nucleotide sequence of SEQ ID NO: 13 can be recited.

To be more specific, when the useful protein gene is a G protein-coupled receptor 7 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a G protein-coupled receptor 7 gene derived from human, and a promoter region located 5′ upstream of the same can be recited, and more concretely, the nucleotide sequence of SEQ ID NO: 14 (corresponding to a nucleotide sequence represented by base No. 75001 to 78000 in the nucleotide sequence described in Genbank Accession No. AC009800) can be recited. In the nucleotide sequence of SEQ ID NO: 14, a nucleotide sequence of exon 1 of a G protein-coupled receptor 7 gene derived from human is represented in base No. 1666 to 2652. Cytosine in the nucleotide sequence represented by CpG which is present in the nucleotide sequence of SEQ ID NO: 14 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1480, 1482, 1485, 1496, 1513, 1526, 1542, 1560, 1564, 1568, 1570, 1580, 1590, 1603, 1613, 1620 and so on in the nucleotide sequence of SEQ ID NO: 14 can be recited.

To be more specific, when the useful protein gene is a G-protein coupled somatostatin and angiotensin-like peptide receptor gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a G-protein coupled somatostatin and angiotensin-like peptide receptor gene derived from human, and a promoter region located 5′ upstream of the same can be recited, and more concretely, the nucleotide sequence of SEQ ID NO: 15 (corresponding to a complementary sequence to a nucleotide sequence represented by base No. 57001 to 60000 in the nucleotide sequence described in Genbank Accession No. AC008971) can be recited. In the nucleotide sequence of SEQ ID NO: 15, a nucleotide sequence of exon 1 of a G-protein coupled somatostatin and angiotensin-like peptide receptor gene derived from human is represented in base No. 776 to 2632. Cytosine in the nucleotide sequence represented by CpG which is present in the nucleotide sequence of SEQ ID NO: 15 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 470, 472, 490, 497, 504, 506, 509, 514, 522, 540, 543, 552, 566, 582, 597, 610, 612 and so on in the nucleotide sequence of SEQ ID NO: 15 can be recited.

To be more specific, when the useful protein gene is a Solute carrier family 6 neurotransmitter transporter noradrenalin member 2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Solute carrier family 6 neurotransmitter transporter noradrenalin member 2 gene derived from human, and a promoter region located 5′ upstream of the same can be recited, and more concretely, the nucleotide sequence of SEQ ID NO: 16 (corresponding to a complementary sequence to a nucleotide sequence represented by base No. 78801 to 81000 in the nucleotide sequence described in Genbank Accession No. ACO26802) can be recited. In the nucleotide sequence of SEQ ID NO: 16, a nucleotide sequence of exon 1 of a Solute carrier family 6 neurotransmitter transporter noradrenalin member 2 gene derived from human is represented in base No. 1479 to 1804. Cytosine in the nucleotide sequence represented by CpG which is present in the nucleotide sequence of SEQ ID NO: 16 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1002, 1010, 1019, 1021, 1051, 1056, 1061, 1063, 1080, 1099, 1110, 1139, 1141, 1164, 1169, 1184 and so on in the nucleotide sequence of SEQ ID NO: 16 can be recited.

In the present invention, the “amount of amplified DNA obtained by (amplifying methylated DNA in the objective DNA region to a detectable level)” means an amount itself after amplification of methylated DNA in the objective region possessed by a genomic DNA contained in the biological specimen, namely, the amount determined in Third step of the present measuring method. For example, when the biological specimen is 1 mL of serum, it means the amount of DNA amplified based on the methylated DNA contained in 1 mL of serum.

The term “methylation rate” in the present invention (particularly, the present methylation rate measuring method) means the numerical value obtained by dividing the amount after amplification of methylated DNA by a total of the amount of methylated DNA after amplification in the objective DNA region possessed by a genomic DNA contained in the biological specimen and the amount of unmethylated DNA after amplification.

The expression “double-stranded DNA containing one or more unmethylated CpG pair in the recognition site of the methylation-sensitive restriction enzyme” in the present invention means double-stranded DNA wherein both of cytosines in one or more CpG pair present in the recognition site of the restriction enzyme in the double-stranded DNA are unmethylated cytosines, namely double-stranded DNA wherein both of cytosine in plus-strand DNA and cytosine in minus-strand DNA corresponding to the plus-strand DNA in one or more CpG pair present in the recognition site of the restriction enzyme in the double-stranded DNA are unmethylated cytosines. Also, the expression “extensionally-formed double-stranded DNA not containing an unmethylated CpG pair in the recognition site of the methylation sensitive restriction enzyme” means double-stranded DNA wherein either one of cytosines in every CpG pair present in the recognition site of the restriction enzyme in double-stranded DNA is methylated, and the other cytosine is unmethylated cytosine, namely double-stranded DNA wherein either one of cytosine in plus-strand DNA and cytosine in minus-strand DNA corresponding to the plus-strand DNA in every CpG pair present in the recognition site of the restriction enzyme in double-stranded DNA is methylated, and the other cytosine is unmethylated cytosine.

In First step of the present measuring method, from a DNA sample derived from a genomic DNA contained in a biological specimen, by causing base-pairing between single-stranded DNA containing an objective DNA region (plus strand), and single-stranded immobilized oligonucleotide having a nucleotide sequence complementary to the objective DNA region, the single-stranded DNA is selected as double-stranded DNA wherein the single-stranded DNA and the single-stranded immobilized oligonucleotide are base-paired.

The “single-stranded immobilized oligonucleotide” in First step of the present measuring method is single-stranded immobilized oligonucleotide having a nucleotide sequence complementary to the entire objective DNA region of the single-stranded DNA (plus strand) containing the objective DNA region, or a nucleotide sequence complementary to a region that is a part of the objective DNA region and contains the 3′-end of the objective DNA region (hereinafter, also referred to as the present immobilized oligonucleotide).

The present immobilized oligonucleotide is used for selecting single-stranded DNA (plus strand) containing an objective DNA region from a DNA sample derived from a genomic DNA contained in a biological specimen. The present immobilized oligonucleotide is preferably 5 to 1000 bases long, and more concretely a base length of 20 to 200 bases is desirable.

The 5′-end side of the present immobilized oligonucleotide may be immobilized to a carrier, while the 3′-end side should be in a free state so as to allow one extension reaction that progresses from the 5′-end to the 3′-end in Second pre step and Step A2 as will be described later. Here, for the expression “the one which can be immobilized to a carrier”, it suffices that the present immobilized oligonucleotide is immobilized to a carrier in selecting the single-stranded DNA (plus strand) containing an objective DNA region, and (1) the one immobilized by binding between the present immobilized oligonucleotide and a carrier in the stage prior to base-paring between the single-stranded DNA (plus strand) and the present immobilized oligonucleotide, or (2) the one immobilized by binding between the present immobilized oligonucleotide and a carrier in the stage after base-paring between the single-stranded DNA (plus strand) and the present immobilized oligonucleotide can be recited.

For obtaining such a structure, the 5′-end of the oligonucleotide may be immobilized to a carrier according to a commonly used genetic engineering operation method or a commercially available kit, device and so on (binding to a solid phase). A concrete exemplary method involves biotinating the 5′-end of the present oligonucleotide, and immobilizing the obtained biotinated oligonucleotide to a support coated with streptavidin (for example, a PCR tube coated with streptavidin, magnetic beads coated with streptavidin and so on).

Also, such a method can be recited that after covalently binding a molecule having an active functional group such as an amino group, an aldehyde group or a thiol group to the 5′-end side of the present oligonucleotide, the product is covalently bound to a support made of glass, silica or heat-resistant plastic having a surface activated with a silane coupling agent or the like, via a spacer, across linker or the like such as five serially-connected triglycerides. Also, a method of chemically synthesizing from the 5′-end side of the present oligonucleotide directly on a support made of glass or silicon is recited.

First step of the present measuring method concretely comprises the following steps (a) to (c), for example, when the present immobilized oligonucleotide is biotinated oligonucleotide.

(a) First, a DNA sample derived from a genomic DNA contained in a biological specimen is added with an annealing buffer and biotinated oligonucleotide (in a free state at the present stage because it will be immobilized by binding between the present immobilized oligonucleotide and a carrier in the stage after base-pairing between the single-stranded DNA (plus strand) and the present immobilized oligonucleotide), to obtain a mixture. Then, the obtained mixture is heated at 95° C. for several minutes so as to make the double-stranded DNA containing an objective DNA region present in the DNA sample derived from a genomic DNA contained in a biological specimen into a single strand. Thereafter, in order to form a double strand of the single-stranded DNA containing an objective DNA region and the biotinated oligonucleotide, the reaction is rapidly cooled to a temperature lower than Tm of the biotinated oligonucleotide by about 10 to 20° C., and kept at this temperature for several minutes.
(b) Thereafter, the reaction is returned to room temperature.
(c) The mixture obtained in the above (b) is added to a support coated with streptavidin, and kept at 37° C. for several minutes, whereby the biotinated oligonucleotide is immobilized on the support coated with streptavidin.

By the way, as described above, in the above (a) to (c), base-pairing between the single-stranded DNA (plus strand) containing an objective DNA region, and the biotinated oligonucleotide is executed in an earlier stage than immobilization of the biotinated oligonucleotide on the support coated with streptavidin, however, the order may be inverted. That is, for example, a mixture is obtained by adding a DNA sample derived from a genomic DNA contained in a biological specimen to biotinated oligonucleotide immobilized to a support coated with streptavidin, and the obtained mixture is heated for several minutes at 95° C. so as to make double-stranded DNA containing an objective DNA region present in the DNA sample derived from a genomic DNA contained in a biological specimen into single strand, and then in order to form a double strand with biotinated oligonucleotide, the reaction may be rapidly cooled to a temperature lower than Tm of the biotinated oligonucleotide by about 10 to 20° C., and kept at this temperature for several minutes.

(d) After immobilizing the biotinated oligonucleotide to the support coated with streptavidin in this manner, the remaining solution is removed and washed (DNA purification).

More concretely, for example, when a PCR tube coated with streptavidin is used, after removing the solution by pipetting or decantation first, a TE buffer of an amount approximately equal to the volume of the biological specimen is added, and thereafter, the TE buffer may be removed by pipetting or decantation. When magnetic beads coated with streptavidin are used, after immobilizing the beads with a magnet, the solution is removed by pipetting or decantation first, and a TE buffer of an amount approximately equal to the volume of the biological specimen is added, and thereafter, the TE buffer may be removed by pipetting or decantation.

Then, by executing these operations several times, the remaining solution is removed and washed (DNA purification).

These operations are important for removing unimmobilized DNA, or DNA floating in the solution digested with the restriction enzyme as will be described later, from the reaction solution. If these operations are inadequate, the DNA floating in the reaction solution will be a template and an unexpected amplification product will be obtained by an amplification reaction. In order to avoid non-specific binding between the support and DNA in the biological specimen, the above operations may be executed while a large amount of DNA having a nucleotide sequence which is completely different from that of the objective region (for example, rat DNA and so on, in the case of a human biological specimen) is added to the biological specimen.

In a preferred embodiment in First step of the present measuring method, in causing base-pairing between single-stranded DNA containing an objective DNA region (plus strand), and single-stranded immobilized oligonucleotide having a nucleotide sequence complementary to the objective DNA region of the single-stranded DNA, the base-pairing is caused in a reaction system containing a divalent cation. More preferably, the divalent cation is a magnesium ion. Here, the “reaction system containing a divalent cation” means a reaction system containing a divalent cation in an annealing buffer used for causing base-paring between the single-stranded DNA (plus strand) and the single-stranded immobilized oligonucleotide, and preferably and concretely, a salt comprising a magnesium ion (for example, MgOAc2, MgCl2 and so on) is contained in a concentration of 1 mM to 600 mM.

In Second step of the present measuring method, after digesting the double-stranded DNA formed in First step with at least one kind of methylation sensitive restriction enzyme, the generated free digest (double-stranded DNA containing one or more unmethylated CpG pair in the recognition site of the methylation sensitive restriction enzyme) is removed.

The “methylation-sensitive restriction enzyme” in Second step of the present measuring method means, for example, a restriction enzyme or the like that does not digest a recognition sequence containing methylated cytosine, but digests only a recognition sequence containing unmethylated cytosine. In other words, in the case of DNA wherein cytosine contained in a recognition sequence inherently recognizable by the methylation sensitive restriction enzyme is methylated, the DNA will not be cleaved even when the methylation sensitive restriction enzyme is caused to act on the DNA. On the other hand, in the case of DNA wherein cytosine contained in a recognition sequence inherently recognizable by the methylation sensitive restriction enzyme is not methylated, the DNA will be cleaved when the methylation sensitive restriction enzyme is caused to act on the DNA. Concrete examples of such methylation sensitive restriction enzymes include HpaII, BstUI, NarI, SacII, and HhaI. The aforementioned methylation sensitive restriction enzyme will not cleave double-stranded DNA containing a CpG pair in a hemimethyl state (namely, double-stranded DNA wherein cytosine in one strand is methylated and cytosine in the other strand is not methylated in the above CpG pair) and this is already revealed by Gruenbaum et al. (Nucleic Acid Research, 9, 2509-2515).

As a method for examining presence/absence of digestion by the methylation sensitive restriction enzyme, concretely, a method of conducting PCR using the aforementioned DNA as a template and a pair of primers capable of amplifying DNA containing cytosine to be analyzed in a recognition sequence, and determining presence/absence of amplification of DNA (amplification product) can be exemplified. When the cytosine to be analyzed is methylated, an amplification product will be obtained. On the other hand, when the cytosine to be analyzed is not methylated, an amplification product will not be obtained. By comparing the amount of amplified DNA in this manner, it is possible to determine the methylated rate of analyzed cytosine.

By the way, in the double-stranded DNA selected in First step, as described above, cytosine contained in the single-stranded immobilized oligonucleotide as a minus strand is in an unmethylated state, while as for cytosine contained in a DNA strand on the plus strand side, whether the state of the double-stranded DNA is unmethylated or not is determined depending on whether cytosine contained in DNA of a genomic DNA contained in a biological specimen is methylated or unmethylated. In other words, when a genomic DNA contained in a biological specimen is methylated, the obtained double-stranded DNA is in a hemimethyl state (the state that is not a non-methyl state, minus strand: an unmethylated state, plus strand: a methylated state), and when a genomic DNA contained in a biological specimen is not methylated, the obtained double-stranded DNA is in a non-methyl state (minus strand: an unmethylated state, plus strand: an unmethylated state). Therefore, by utilizing the characteristic that the aforementioned methylation sensitive restriction enzyme does not cleave double-stranded DNA in a hemimethyl state, it is possible to distinguish whether cytosine in at least one CpG pair present in the recognition site of the methylation sensitive restriction enzyme in a genomic DNA in the biological specimen is methylated or not. That is, by digesting with the methylation sensitive restriction enzyme, if cytosine in at least one CpG pair present in the recognition site of the methylation sensitive restriction enzyme in a genomic DNA contained in the biological specimen is not methylated, the double-stranded DNA is in a non-methyl state, and cleaved by the methylation sensitive restriction enzyme. If cytosine in every CpG pair present in the recognition site of the methylation sensitive restriction enzyme in a genomic DNA contained in the biological specimen is methylated, the double-stranded DNA is in a hemimethyl state, and will not be cleaved by the methylation sensitive restriction enzyme. Therefore, by executing PCR using a pair of primers capable of amplifying the objective DNA region as will be described later after executing the digestion treatment, an amplification product by PCR will not be obtained when cytosine in at least one CpG pair present in the recognition site of the restriction enzyme in a genomic DNA contained in the biological specimen is not methylated, whereas an amplification product by PCR will be obtained when cytosine in every CpG pair present in the recognition site of the methylation-sensitive restriction enzyme in a genomic DNA contained in the biological specimen is methylated.

Concretely, Second step of the present measuring method may be executed, for example, in the following manner when the present immobilized oligonucleotide is biotinated oligonucleotide. Double-stranded DNA generated in First step is added respectively with 3 μL of an optimum 10× buffer (330 mM Tris-Acetate pH 7.9, 660 mM KOAc, 100 mM MgOAc2, 5 mM Dithiothreitol), 3 μL of a 1 mg/mL aqueous BSA solution, and each 1.5 μL of a methylation sensitive restriction enzyme HpaII or HhaI (10 U/μL) and so on, and the mixture is added with sterilized ultrapure water to make the liquid volume 30 μL, and incubated at 37° C. for 1 hour to 3 hours.

After digesting the double-stranded DNA formed in First step with at least one kind of methylation sensitive restriction enzyme, the generated free digest (double-stranded DNA containing one or more unmethylated CpG pair in the recognition site of the methylation-sensitive restriction enzyme) is removed and washed (DNA purification).

More concretely, for example, when a PCR tube coated with streptavidin is used, after removing the solution by pipetting or decantation first, a TE buffer of an amount approximately equal to the volume of the biological specimen is added, and thereafter, the TE buffer may be removed by pipetting or decantation. When magnetic beads coated with streptavidin are used, after immobilizing the beads with a magnet, the solution is removed by pipetting or decantation first, and a TE buffer of an amount approximately equal to the volume of the biological specimen is added, and thereafter, the TE buffer may be removed by pipetting or decantation.

Then, by executing such an operation several times, the digest (double-stranded DNA containing one or more unmethylated CpG pair in the recognition site of the restriction enzyme) is removed and washed (DNA purification).

As a fear in a digestion treatment with the methylation-sensitive restriction enzyme in Second step of the present measuring method, the possibility that the recognition sequence containing unmethylated cytosine is not completely digested (so called “DNA remaining undigested”) is recited. When such a possibility is problematic, the objective DNA region has at least one recognition site of the methylation sensitive restriction enzyme, and as many as possible recognition sites would be preferred because it is possible to suppress the “DNA remaining undigested” as much as possible by presence of abundant recognition sites of the methylation sensitive restriction enzyme.

Therefore, when a treatment with a plurality of methylation sensitive restriction enzymes is executed in Second step of the present measuring method, the treatment may be, for example, concretely executed in the following manner. Double-stranded DNA selected in First step is added respectively with 3 μL of an optimum 10× buffer (330 mM Tris-Acetate pH 7.9, 660 mM KOAc, 100 mM MgOAc2, 5 mM dithiothreitol), 3 μl of a 1 mg/mL aqueous BSA solution, and each 1.5 μL of a methylation sensitive restriction enzyme HpaII or HhaI (10 U/μL) and so on, and the mixture is added with sterilized ultrapure water to make the liquid volume 30 μL, and incubated at 37° C. for 1 hour to 3 hours. Thereafter, the remaining solution is removed and washed (DNA purification) by pipetting or decantation according to the operation similar to that described above. More concretely, for example, when a PCR tube coated with streptavidin is used, after removing the solution by pipetting or decantation first, a TE buffer of an amount approximately equal to the volume of the biological specimen is added, and thereafter, the TE buffer may be removed by pipetting or decantation. When magnetic beads coated with streptavidin are used, after immobilizing the beads with a magnet, the solution is removed by pipetting or decantation first, and a TE buffer of an amount approximately equal to the volume of the biological specimen is added, and thereafter, the TE buffer may be removed by pipetting or decantation.

Then, by executing such operations several times, the remaining solution is removed and washed (DNA purification).

In the present measuring method or a methylation rate measuring method as will be described later, one preferable exemplary embodiment is that “a DNA sample derived from a genomic DNA contained in a biological specimen” is a DNA sample digested in advance with a restriction enzyme whose recognition cleavage site excludes the objective DNA region possessed by the genomic DNA. Here, when a genomic DNA contained in a biological specimen (template DNA) is selected with the use of single-stranded immobilized oligonucleotide, shorter template DNA is more likely to be selected, and when the objective region is amplified by PCR, shorter template DNA is more preferred. Therefore, a digestion treatment may be executed while using a restriction enzyme whose recognition cleavage site excludes the objective DNA region directly on the DNA sample derived from a genomic DNA contained in a biological specimen. As a method of digesting with a restriction enzyme whose recognition cleavage site excludes the objective DNA region, a commonly used restriction enzyme treatment method may be used.

One exemplary preferable embodiment is that “a DNA sample derived from a genomic DNA contained in a biological specimen” is a DNA sample digested with at least one kind of methylation sensitive restriction enzyme.

These embodiments are preferred because the methylation amount can be determined accurately by digesting the biological specimen itself in advance with a restriction enzyme as described above. Such a method is useful for avoiding the “DNA remaining undigested” as described above.

As a method of digesting a sample derived from a genomic DNA contained in a biological specimen with the methylation sensitive restriction enzyme, when the biological specimen is a genomic DNA itself, the method similar to that described above is preferred, and when the biological specimen is a tissue lysate, a cell lysate or the like, a digestion treatment may be executed using a large excess of methylation sensitive restriction enzyme, for example, a methylation sensitive restriction enzyme in an amount of 500 times (10 U) or more with respect to 25 ng of the DNA amount, according to a similar method as described above.

Third step of the present measuring method comprises as pre steps of each of the following regular steps:

Step (First pre step) of temporarily separating the formed double-stranded DNA (formed double-stranded DNA not containing an unmethylated CpG pair in a recognition site of the methylation-sensitive restriction enzyme) which is an undigested substance obtained in Second step, in a single-stranded state, as a pre step of each of the following regular steps;

Step (Second pre step) comprising:

Step (Second (A) pre step) of causing base-pairing between the generated free single-stranded DNA (plus strand) and the single-stranded immobilized oligonucleotide, thereby selecting the generated free single-stranded DNA, and forming double-stranded DNA made up of the selected single-stranded DNA and the single-stranded immobilized oligonucleotide that are base-paired, and

Step (Second (B) pre step) of making the double-stranded DNA formed in Step (Second (A) pre step) into double-stranded DNA extended and formed from the selected single-stranded DNA by extending a primer once using the selected single-stranded DNA as a template and the single-stranded immobilized oligonucleotide as a primer; and

Step (Third pre step) of temporarily separating the double-stranded DNA (extensionally-formed double-stranded DNA not containing an unmethylated CpG pair in a recognition site of the methylation-sensitive restriction enzyme) extended and formed in Second pre step into a single-stranded DNA (plus strand) and a single-stranded DNA (minus strand), and as regular steps,

(a) Step A (a regular step) comprising:

Step A1 of causing base-paring between the generated a single-stranded DNA (plus strand) and the single-stranded immobilized oligonucleotide (minus strand), thereby selecting the single-stranded DNA, and

Step A2 of extending and forming the DNA in a single-stranded state as double-stranded DNA by one extension of a primer using the single-stranded DNA selected in Step A1 as a template and the single-stranded immobilized oligonucleotide as a primer, (b) Step B (a regular step) of making the single-stranded DNA into extensionally-formed double-stranded DNA by one extension of oligonucleotide by using the generated single-stranded DNA (minus strand) as a template, and oligonucleotide (a reverse primer) having a nucleotide sequence (plus strand) complementary to a partial nucleotide sequence (minus strand) of a nucleotide sequence possessed by the DNA in a single-stranded state (minus strand), wherein the partial nucleotide sequence (minus strand) is positioned on the 3′-end side than the 3′-end of the nucleotide sequence (minus strand) complementary to the nucleotide sequence (plus strand) of the objective DNA region, and is not usable in an extension reaction using the single-stranded immobilized oligonucleotide as a template, as an extension primer,

and the methylated DNA in the objective DNA region is amplified to a detectable level by repeating each regular step of Third step after temporarily separating the extensionally-formed double-stranded DNA obtained in each regular step into a single-stranded state, and the amplified DNA is quantified.

In Third step of the present measuring method, first, as First pre step among pre steps of respective regular steps, double-stranded DNA (extensionally-formed double-stranded DNA not containing an unmethylated CpG pair in the recognition site of the methylation-sensitive restriction enzyme) which is an undigested substance obtained in Second step is temporarily separated in a single-stranded state. Concretely, for example, double-stranded DNA which is an undigested substance obtained in Second step (double-stranded DNA not containing an unmethylated CpG pair in the recognition site of the methylation-sensitive restriction enzyme) is added with an annealing buffer to obtain a mixture. Then, the obtained mixture is heated for several minutes at 95° C. Thereafter, Second (A) pre step in Second pre step may be executed, for example, in conformance with First step concretely, and double-stranded DNA wherein single-stranded DNA in a methylation state and the present immobilized oligonucleotide are base-paired is formed. This allows selection of the double-stranded DNA.

Second (B) pre step may be concretely executed in the following manner, for example, when the present immobilized oligonucleotide is biotinated oligonucleotide.

Single-stranded DNA in a methylation state selected as double-stranded DNA and single-stranded immobilized oligonucleotide are added with 17.85 μL of sterilized ultrapure water, 3 uL of an optimum 10× buffer (100 mM Tris-HCl pH 8.3, 500 mM KCl, 15 mM MgCl₂), 3 μL of 2 mM dNTP, and 6 μL of 5 N betaine, and then the resultant mixture is added with 0.15 μL of AmpliTaq (a kind of DNA polymerase: 5 U/μL) to make the liquid volume 30 μL, followed by 2-hour incubation at 37° C. Thereafter, the incubated solution is removed by pipetting or decantation, and a TE buffer of an amount approximately equal to the volume of the biological specimen is added, and then the TE buffer may be removed by pipetting or decantation.

More concretely, for example, when a PCR tube coated with streptavidin is used, after removing the solution by pipetting or decantation first, a TE buffer of an amount approximately equal to the volume of the biological specimen is added, and then the TE buffer may be removed by pipetting or decantation. When magnetic beads coated with streptavidin are used, after immobilizing the beads with a magnet, the solution is removed by pipetting or decantation first, and a TE buffer of an amount approximately equal to the volume of the biological specimen is added, and then the TE buffer may be removed by pipetting or decantation.

Then, by executing these operations several times, the remaining solution is removed and washed (DNA purification).

In Third pre step, extensionally-formed double-stranded DNA obtained in Second (B) pre step (extensionally-formed double-stranded DNA not containing an unmethylated CpG pair in the recognition site of the methylation-sensitive restriction enzyme) is temporarily separated into a single-stranded DNA (plus strand) and a single-stranded DNA (minus strand). Concretely, for example, double-stranded DNA obtained in Second (B) pre step (double-stranded DNA not containing an unmethylated CpG pair in the recognition site of the methylation-sensitive restriction enzyme) is added with an annealing buffer, to obtain a mixture. Then, the obtained mixture is heated at 95° C. for several minutes. Then, the following regular steps are conducted.

(i) The reaction is rapidly cooled to a temperature lower than Tm of single-stranded immobilized oligonucleotide (minus strand) by about 10 to 20° C., and kept at this temperature for several minutes for annealing the generated a single-stranded DNA (plus strand) with the single-stranded immobilized oligonucleotide (minus strand).
(ii) Thereafter, the reaction is returned to room temperature (Step A1 in Step A).
(iii) By one extension of a primer using a single-stranded DNA selected in the above (i) as a template and the single-stranded immobilized oligonucleotide as a primer, the a single-stranded DNA is extended and formed as double-stranded DNA (that is, Step A2 in Step A). Concretely, for example, it may be practiced in conformance with the operation method in an extension reaction as will be described later or in Second (B) pre step of the present measuring method as described above.
(iv) The a single-stranded DNA is made into extensionally-formed double-stranded DNA by one extension of oligonucleotide by using the generated DNA in a single-stranded state (minus strand) as a template, and oligonucleotide (a reverse primer) having a nucleotide sequence (plus strand) complementary to a partial nucleotide sequence (minus strand) of a nucleotide sequence possessed by the DNA in a single-stranded state (minus strand), wherein the partial nucleotide sequence (minus strand) is positioned on the 3′-end side than the 3′-end of the nucleotide sequence (minus strand) complementary to the nucleotide sequence (plus strand) of the objective DNA region, and is not usable in an extension reaction using the single-stranded immobilized oligonucleotide as a template, as an extension primer (that is, Step B). Concretely, for example, similarly to the above (iii), it may be practiced in conformance with the operation method in an extension reaction as will be described later or in Second (B) pre step of the present measuring method as described above.
(v) The methylated DNA in the objective DNA region is amplified to a detectable level by repeating each regular step of Third step (namely Step A and Step B) after temporarily separating the extensionally-formed double-stranded DNA obtained in each regular step into a single-stranded state, and the amplified DNA is quantified. Concretely, for example, similarly to the above description, it may be practiced in conformance with the operation method as will be described later or in Second (B) pre step, Step A and Step B in Third step of the present measuring method as described above.

By using an extension primer which is unable to utilize single-stranded immobilized oligonucleotide as a template for extension reaction, as a reverse primer, it is possible to specifically amplify double-stranded DNA (double-stranded DNA not containing an unmethylated CpG pair in the recognition site of the methylation-sensitive restriction enzyme) extended and formed in Second (B) pre step without amplifying single-stranded (immobilized) oligonucleotide not extended and formed in Third step.

In Third step, concretely the reaction starting from First pre step and up to the regular step may be executed as a single PCR reaction. Alternatively, from First pre step to Third pre step, each reaction may be independently executed, and only a regular step may be executed as a PCR reaction.

As a method of amplifying an objective DNA region (namely, an objective region) after digestion with the methylation sensitive restriction enzyme, for example, PCR may be used. Since the present immobilized oligonucleotide can be used as one of the primers in amplifying the objective region, an amplification product can be obtained by conducting PCR while adding only the other of the primers, and its amplification product is also immobilized. At this time, by using a primer labeled in advance with fluorescence or the like and utilizing the label as an index, it is possible to evaluate presence or absence of an amplification product without executing a burdensome operation such as electrophoresis. As a PCR reaction solution, for example, a reaction solution obtained by mixing DNA obtained in Second step of the present measuring method with 0.15 μL of a 50 μM primer solution, 2.5 μL of 2 mM dNTP, 2.5 μL of a 10× buffer (100 mM Tris-HCl pH 8.3, 500 mM KCl, 20 mM MgCl₂, 0.01% Gelatin), and 0.2 μL of AmpliTaq Gold (one kind of thermostable DNA polymerase: 5 U/μL), and adding sterilized ultrapure water to make the liquid volume 25 μL can be recited.

Since an objective DNA region (namely, an objective region) often has a GC rich nucleotide sequence, the reaction may sometimes be executed while adding an appropriate amount of betaine, DMSO or the like. In one exemplary reaction conditions, the reaction solution as described above is retained at 95° C. for 10 minutes, and then a cycle made up of 30 seconds at 95° C., 30 seconds at 55 to 65° C., and 30 seconds at 72° C. is repeated 30 to 40 cycles. After conducting such PCR, the obtained amplification product is detected. For example, when a primer labeled in advance is used, after executing washing and purification operations similar to those as described above, an amount of an immobilized fluorescent label may be measured. When PCR is conducted using a normal primer that is not labeled, a probe or the like that is labeled with gold colloid particles, fluorescence or the like is caused to anneal, and detection may be achieved by measuring an amount of the probe bound to the objective region. Also, in order to determine an amount of the amplification product more accurately, for example, a real-time PCR method may be used. The real-time PCR is a method in which PCR is monitored in real time, and the obtained monitor result is analyzed kinetically, and is known as a high-accuracy quantitative PCR method capable of detecting a very small difference as small as twice the gene amount. As such a real-time PCR method, for example, a method using a probe such as a template-dependent nucleic acid polymerase probe, a method of using an intercalator such as SYBR-Green and the like can be recited. As an apparatus and a kit for the real-time PCR method, those commercially available may be used. As described above, detection may be executed by any method well-known heretofore without any particular limitation. Such methods make it possible to conduct the operations up to detection without changing the reaction container.

It is also possible to amplify the objective region by using biotinated oligonucleotide having the same nucleotide sequence as the single-stranded immobilized oligonucleotide as one of the primers, or new biotinated oligonucleotide designed on the 3′-end side than the single-stranded immobilized oligonucleotide as one of the primers, and a primer on the complementary side. In this case, since the obtained amplification product is immobilized if there is a support coated with streptavidin, for example, when PCR is executed in a PCR tube coated with streptavidin, use of a labeled primer as described above will facilitate detection of the amplification product because it will be immobilized inside the tube. When the foregoing single-stranded immobilized oligonucleotide is immobilized by covalent bonding or the like, the solution containing an amplification product obtained by PCR may be transferred to a container where a support coated with streptavidin is present, and the amplification product may be immobilized. Detection may be executed in the manner as described above. The complementary side primer for amplifying an objective region should be a primer that is capable of amplifying an objective region having at least one recognition site of the methylation-sensitive restriction enzyme and not containing the recognition site. The reason is as follows. When only the most 3′-end side recognition site of the methylation sensitive restriction enzyme of a DNA chain (newly-generated strand) on the present immobilized oligonucleotide side of double-stranded DNA obtained by selection and one extension reaction is not methylated, only that part will be digested by the methylation-sensitive restriction enzyme. Even if a washing operation is conducted as described above after digestion, double-stranded DNA in which a part of the 3′-end in the newly-generated strand is lost remains immobilized. When the complementary side primer contains the most 3′-end side recognition site of the methylation sensitive restriction enzyme, several bases on the 3′-end side of the primer anneal with several bases on the 3′-end side of the newly-generated strand, so that the objective region can be amplified by PCR.

The present invention also includes a modified method additionally comprising a step of adding single-stranded oligonucleotide (minus strand) having a nucleotide sequence complementary to a part of the 3′-end of single-stranded DNA containing an objective DNA region (plus strand) and is in a free state into a reaction system (Additional pre step), in the previous operation stage or the post operation stage of First pre step in Third step of the present measuring method, or in the previous operation stage or the post operation stage of Third pre step in Third step.

### (Modified Method 1)

A method further comprising in a previous operation stage of First pre step in Third step of the present measuring method:

a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and

additionally comprising one of the following steps as a respective regular step of Third step of the present measuring method:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

### (Modified Method 2)

A method further comprising in a post operation stage of First pre step in Third step of the present measuring method:

a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and

a step (additional re-pre step) of temporarily separating double-stranded DNA (extensionally-formed double-stranded DNA not containing an unmethylated CpG pair in the recognition site of the methylation-sensitive restriction enzyme) which is an undigested substance obtained through First pre step and Additional pre step into a single-stranded state, and

additionally comprising the following one step as a respective regular step of Third step of the present measuring method:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer (hereinafter, also referred to as the present methylation rate measuring method).

### (Modified Method 3)

A method further comprising in a previous operation stage of Third pre step in Third step of the present measuring method:

a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and

additionally comprising the following one step as a respective regular step of Third step of the present measuring method:

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

### (Modified Method 4)

A method further comprising in a post operation stage of Third pre step in Third step of the present measuring method:

a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and

a step (additional re-pre step) of temporarily separating double-stranded DNA (extensionally-formed double-stranded DNA not containing an unmethylated CpG pair in the recognition site of the methylation-sensitive restriction enzyme) which is an undigested substance obtained through First pre step and Additional pre step into a single-stranded state, and

additionally comprising the following one step as a respective regular step of Third step of the present measuring method (hereinafter, also referred to as the present methylation rate measuring method):

(c) Step C (a regular step) comprising:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

In the modified method, it is possible to readily improve the amplification efficiency of the objective DNA region in Third step by, for example, adding “single-stranded oligonucleotide (minus strand) having a nucleotide sequence complementary to a part of the 3′-end of single-stranded DNA containing an objective DNA region (plus strand) and is in a free state” to a reaction system externally. The single-stranded oligonucleotide (minus strand) added into the reaction system in Additional pre step may have the same nucleotide sequence as that of the single-stranded immobilized oligonucleotide, or may have a shorter nucleotide sequence or a longer nucleotide sequence insofar as it is single-stranded oligonucleotide having a nucleotide sequence complementary to a part of the 3′-end of single-stranded DNA (not containing the objective DNA region) and has the 5′-end which is as same as the single-stranded immobilized oligonucleotide and in a free state. However, in the case of a nucleotide sequence longer than the single-stranded immobilized oligonucleotide, it is important to be single-stranded oligonucleotide in a free state which is unavailable in reaction of extending an extension primer by using the reverse primer (plus strand) as an extension primer, and the single-stranded oligonucleotide (minus strand) as a template.

While an explanation was made for the case where PCR is executed while using immobilized oligonucleotide as one of primers and adding the other of primers in amplifying the objective region, when other methods for detecting the objective product (for example, an analytical method capable of comparing the amount of each amplification product obtained by PCR) are executed, PCR may be executed while adding a pair of primers rather than using immobilized oligonucleotide as either one of primers in amplifying the objective region as described above. After conducting such PCR, an amount of the obtained amplification product is determined.

Third step of the present measuring method has repeated steps, and for example, “generated single-stranded DNA (plus strand)” in Step A1 means “generated “free” DNA in a single-stranded state (plus strand)” both in first operation of Third step and in second or later repeated operation of Third step.

In Step B, “generated single-stranded DNA (minus strand)” means “generated “immobilized” DNA in a single-stranded state (plus strand)” both in first operation of Third step and in second or later repeated operation of Third step. However, when Third step further has Step C additionally, it means “generated “immobilized” DNA in a single-stranded state (plus strand)” in first operation of Third step, while it means both “generated “immobilized” DNA in a single-stranded state (plus strand)” and “generated “free” DNA in a single-stranded state (plus strand)” in second or later repeated operation of Third step.

In the case of Step A, “extensionally-formed double-stranded DNA” obtained in each regular step of Third step means “extensionally-formed double-stranded DNA not containing an unmethylated CpG pair in the recognition site of the methylation-sensitive restriction enzyme” in first operation of Third step, while it means both “extensionally-formed double-stranded DNA not containing an unmethylated CpG pair in the recognition site of the methylation-sensitive restriction enzyme” and “extensionally-formed double-stranded DNA containing an unmethylated CpG pair in the recognition site of the methylation-sensitive restriction enzyme” in second or later repeated operation of Third step. In the case of Step B, it means “extensionally-formed double-stranded DNA wherein every one is an unmethylated CpG pair in the recognition site of the methylation-sensitive restriction enzyme” both in first operation of Third step and in second or later repeated operation of Third step.

The same applies to the case where Third step further has Step C additionally.

In the case where Third step further has Step C additionally, “generated single-stranded DNA (plus strand)” in Step C1 means “generated “free” DNA in a single-stranded state (plus strand)” both in first operation of Third step and in second or later repeated operation of Third step.

The present invention also includes a method of measuring a methylation rate (namely, the present methylation rate measuring method) further comprising the following two steps additionally as steps of the present measuring method:

(4) Fourth step of amplifying DNA (total amount of methylated DNA and unmethylated DNA) of the objective DNA region to a detectable level by conducting Third step in the present measuring method (including the aforementioned modified method) without conducting Second step in the present measuring method (including the aforementioned modified method) after conducting First step in the present measuring method (including the aforementioned modified method), and quantifying the amplified DNA; and
(5) Fifth step of calculating a rate of methylated DNA in the objective DNA region based on a difference obtained by comparing the DNA amount quantified by Third step in the present measuring method (including the aforementioned modified method), and the DNA amount quantified in Fourth step.

The methylation rate measuring method may be used in the following situations.

It is known that DNA methylation abnormality occurs in various diseases (for example, cancer), and it is believed that the degree of various diseases can be measured by detecting this DNA methylation abnormality.

For example, when there is a DNA region where methylation occurs at 100% in a genomic DNA contained in a specimen derived from a diseased organism, and the present measuring method or the present methylation rate measuring method is executed for the DNA region, the amount of methylated DNA would increase, and for example, when there is a DNA region where methylation does not occur at 100% in a genomic DNA contained in a specimen derived from a diseased organism, and the present measuring method or the present methylation rate measuring method is executed for the DNA region, the amount of methylated DNA would be approximately 0. For example, when there is a DNA region where the methylation rate is low in a genomic DNA contained in a specimen derived from a healthy organism, and a DNA region where the methylation rate is high in a genomic DNA contained in a specimen derived from a diseased organism, and the present measuring method or the present methylation rate measuring method is executed for the DNA region, the amount of methylated DNA would be approximately 0 for a healthy subject, and a significantly higher value than that of a healthy subject would be exhibited by a disease subject, so that the “degree of disease” can be determined based on this difference in value. The “degree of disease” used herein has the same meaning as those commonly used in this field of art, and concretely means, for example, malignancy when the biological specimen is a cell, and means, for example, abundance of disease cells in the tissue when the biological specimen is a tissue. Further, when the biological specimen is plasma or serum, it means the probability that the individual has a disease. Therefore, the present measuring method or the present methylation rate measuring method makes it possible to diagnose various diseases by examining methylation abnormality.

Restriction enzymes, primers or probes that can be used in various methods for measuring a methylated DNA amount in an objective region, and for measuring a methylation rate in the present measuring method or the present methylation rate measuring method are useful as reagents of a detection kit. The present invention also provides a detection kit containing these restriction enzymes, primers or probes as reagents, and a detection chip in which these primers or probes are immobilized on a carrier, and a scope of the present measuring method or the present methylation rate measuring method includes use in the form of the detection kit or the detection chip as described above utilizing the substantial principle of such a method.

### EXAMPLES

In the following, the present invention will be explained in detail by way of examples, however, the present invention will not be limited to these examples.

### Example 1

A mammal-derived colorectal adenocarcinoma cell line Caco-2 (ATCC NO. HTB-37) purchased from ATCC was cultured to confluent in a special medium for the cell line described in an ATCC catalogue, to obtain about 1×10⁷ cells each. The obtained cells were added with 10-times volume of a SEDTA buffer [10 mM Tris-HCl pH 8.0, 10 mM EDTA pH 8.0, 100 mM NaCl] and homogenized.

After adding the obtained mixture with 500 pg/mL of proteinase K (Sigma) and sodium dodecyl sulfate in a concentration of 1% (w/v), the mixture was shaken at 55° C. for about 16 hours.

After completion of shaking, the mixture was subjected to extraction with phenol [saturated in 1 M Tris-HCl, pH 8.0)]/chloroform. The aqueous layer was collected, added with NaCl in a concentration of 0.5 N, subjected to ethanol precipitation, and the generated precipitate (a genomic DNA) was collected. The collected precipitate was dissolved in a TE buffer (10 mM Tris, 1 mM EDTA, pH 8.0), added with RNase A (Sigma) in a concentration of 40 μg/mL, and incubated at 37° C. for 1 hour. The incubated mixture was subjected to phenol/chloroform extraction. The aqueous layer was collected, added with NaCl in a concentration of 0.5 N, and subjected to ethanol precipitation, to collect precipitates (a genomic DNA). By rinsing the collected precipitates with 70% ethanol, the genomic DNA was obtained.

By conducting PCR using the obtained genomic DNA as a template, and using the following primers and reaction conditions, a DNA fragment (hereinafter, denoted by a DNA fragment X2 having a nucleotide sequence represented by SEQ ID NO: 18. A sequence corresponding to base No. 76477 to 77002 in the GPR7 sequence shown in Genbank Accession No. AC009800 or the like) used as a test sample was amplified.

| | | |
|---|---|---|
| PF3: | 5′-GTCCGCGGCGACATTGGG-3′ | (SEQ ID NO: 19) |
| PR3: | 5′-CGATGAGCTTGCACATGAGCT-3′ | (SEQ ID NO: 20) |

As a reaction solution of PCR, 2.5 ng of the genomic DNA which is a template, a solution of a primer having the nucleotide sequence of SEQ ID NO: 19 prepared into 3 μM, 2.5 μL of a solution of a primer having the nucleotide sequence of SEQ ID NO: 20, each 2.5 μL of 2 mM of dNTPs, 2.5 μL of a 10× buffer (100 mM Tris-HCl pH 8.3, 500 mM KCl, 15 mM MgCl₂, 0.01% Gelatin) and 0.125 μL of thermostable DNA polymerase (AmpliTaq Gold, 5 U/μL) were mixed, and added with sterilized ultrapure water to make the liquid volume 25 μL. After keeping the reaction solution at 95° C. for 10 minutes, a cycle consisting of 30 seconds at 95° C., 60 seconds at 59° C., and 45 seconds at 72° C. was repeated 50 times to conduct PCR.

After conducting PCR, DNA amplification was examined by 1.5% agarose gel electrophoresis, and an objective DNA fragment (526 bp, DNA fragment X2) was cut out, and purified by using QIAGEN QIAquick Gel Extraction Kit (available from QIAGEN).

A part of the obtained DNA fragment X2 was treated with a methylation enzyme SssI (available from NEB), to obtain a DNA fragment (hereinafter, denoted by a DNA fragment Y2) in which every 5′-CG-3′ was methylated. Also in this case, likewise the above case, amplification was examined by 1.5% agarose gel electrophoresis, and a DNA fragment (526 bp, DNA fragment Y2) was cut out and purified by using QIAGEN QIAquick Gel Extraction Kit (available from QIAGEN).

As oligonucleotide to be annealed with the DNA fragment X2 and the DNA fragment Y2, oligonucleotide B1 (80 bp) having the nucleotide sequence of SEQ ID NO: 21, labeled with biotin at its 5′-end was synthesized.

<Biotin-labeled oligonucleotide>

| | | |
|---|---|---|
| B1: | 5′-GACAACGCCTCGTTCTCGG-3′ | (SEQ ID NO: 21) |

Each of respective solutions of the DNA fragment X2 and the DNA fragment Y2 (25 pg/mL aqueous solution, 10 μL) was added with 1 μL of 5 μM biotinated oligonucleotide B1, and 10 μL of an annealing buffer (0.5M Church's phosphate buffer, 7% SDS, 1 mM EDTA aqueous solution), to obtain a mixture.

Then, the obtained mixture was heated at 95° C. for 5 minutes. Then, the mixture was rapidly cooled to 50° C., and kept at this temperature for 5 minutes. Then the mixture was kept at 37° C. for 5 minutes, and returned to room temperature, to allow base-pairing between single-stranded DNA (the DNA fragment X2 or the DNA fragment Y2) containing the objective DNA region, and biotinated oligonucleotide (two were prepared for each of the DNA fragment X2 and the DNA fragment Y2).

Then, the mixture prepared in the manner as describe above was introduced into a PCR tube coated with streptavidin, and kept at 37° C. for 5 minutes, whereby the biotinated oligonucleotide was immobilized on the support coated with streptavidin (these correspond to First step of the present measuring method).

Then, after removing the solution from the PCR tube, 100 μL of a TE buffer was added, and the TE buffer was removed by pipetting. This operation was repeated another two times.

The double-stranded DNA thus obtained was subjected to the following two kinds of treatments.

Group A (untreated group): The double-stranded DNA prepared above was added with 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL.
Group B (HpaII and HhaI digestion treatment group): The double-stranded DNA prepared above was added with each 15 U of HpaII and HhaI, 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL.

After incubating each reaction solution at 37° C. for 1 hour (digestion treatment), the supernatant was removed, 100 μL of a TE buffer was added, and the TE buffer was removed by pipetting (these correspond to Second step of the present measuring method).

Then, using the obtained undigested substance as a template, PCR was conducted using the following primers and reaction conditions. If the objective DNA region is methylated, DNA (having the nucleotide sequence of SEQ ID NO: 22, a sequence corresponding to base No. 76669 to 76835 in the GPR7 sequence shown in Genbank Accession No. AC009800 and so on) would be amplified.

| | | |
|---|---|---|
| PF4: | 5′-GACAACGCCTCGTTCTCGG-3′ | (SEQ ID NO: 23) |
| PR4: | 5′-GCGGAGTTGCCCGCCAGA-3′ | (SEQ ID NO: 24) |

As a reaction solution of PCR, the genomic DNA which is a template was mixed with each 2.5 μL of a solution of a primer having the nucleotide sequence of SEQ ID NO: 23 and a solution of a primer having the nucleotide sequence of SEQ ID NO: 24 prepared into 3 μM, each 2.5 μL of each 2 mM of dNTPs, 2.5 μL of a 10× buffer (100 mM Tris-HCl pH 8.3, 500 mM KCl, 15 mM MgCl₂, 0.01% Gelatin), 0.125 μL of 5 U/μL of thermostable DNA polymerase (AmpliTaq Gold), and 5 μL of a 5 N aqueous betaine solution, and the mixture was added with sterilized ultrapure water to make the liquid volume 25 μL. PCR was conducted in such a condition that the reaction solution was kept at 95° C. for 10 minutes, followed by 37 cycles each including 30 seconds at 95° C., 60 seconds at 59° C. and 45 seconds at 72° C.

After conducting PCR, amplification was examined by 1.5% agarose gel electrophoresis. The result was as follows (these correspond to Third step of the present measuring method).

As for the DNA fragment X2, in the case of Group A (untreated group), amplification was observed, and its amplification product was obtained. On the other hand, in the case of Group B (HpaII, HhaI treated group), amplification was not observed, and its amplification product was not obtained. On the other hand, as for the DNA fragment Y2, amplification was observed and its amplification product was obtained both in the cases of Group A (untreated group) and Group B (HpaII, HhaI treated group).

From the above, it was confirmed that single-stranded DNA containing the objective DNA region can be selected, and only methylated DNA can be amplified to a detectable level without amplifying unmethylated DNA in the objective DNA region, and an amount of amplified DNA can be quantified.

### Example 2

Using the DNA fragment X2 and the DNA fragment Y2 obtained in Example 1, the following test was conducted.

By adding each of the DNA fragment X2 and the DNA fragment Y2 individually into 1 mL of rat serum so that each of these fragment was in a proportion of 25 pg/mL, a serum solution was obtained. The serum solution (DNA fragment 25 pg/mL aqueous solution, 10 μL) was added with 1 μL of 5 μM biotinated oligonucleotide B1, and 10 μL of an annealing buffer (0.5 M Church's phosphate buffer, 7% SDS, 1 mM EDTA aqueous solution), to obtain a mixture.

Then, the obtained mixture was heated at 95° C. for 5 minutes. Then the mixture was rapidly cooled to 50° C., and kept at this temperature for 5 minutes. After keeping at 37° C. for 5 minutes, the mixture was returned to room temperature, to cause base-pairing between single-stranded DNA containing the objective DNA region and biotinated oligonucleotide (each 6 samples were prepared for the DNA fragment X2 and the DNA fragment Y2).

Then, the mixture prepared in the manner as describe above was introduced into a PCR tube coated with streptavidin, and kept at 37° C. for 5 minutes, whereby the biotinated oligonucleotide was immobilized on the support coated with streptavidin (these correspond to First step of the present measuring method).

Then, after removing the solution from the PCR tube, 100 μL of a TE buffer was added, and the TE buffer was removed by pipetting. This operation was repeated another two times.

The double-stranded DNA thus selected and obtained was subjected to the following two kinds of treatments.

Group A (untreated group): The double-stranded DNA prepared above was added with 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL (each 3 samples were prepared).
Group B (HpaII and HhaI digestion treatment group): The double-stranded DNA prepared above was added with each 15 U of HpaII and HhaI, 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL (each 3 samples were prepared).

After incubating each reaction solution at 37° C. for 1 hour (digestion treatment), the supernatant was removed, 100 μL of a TE buffer was added, and the TE buffer was removed by pipetting (these correspond to Second step of the present measuring method).

Then, from the obtained undigested substance, PCR was conducted using the following primers and reaction conditions. If the objective DNA region is methylated, DNA (SEQ ID NO: 22, a sequence corresponding to base No. 76669 to 76835 in the GPR7 sequence shown in Genbank Accession No. AC009800 and so on) would be amplified.

| | | |
|---|---|---|
| PF4: | 5′-GACAACGCCTCGTTCTCGG-3′ | (SEQ ID NO: 23) |
| PR4: | 5′-GCGGAGTTGCCCGCCAGA-3′ | (SEQ ID NO: 24) |

As a reaction solution of PCR, the genomic DNA which is a template was mixed with each 2.5 μL of a solution of a primer having the nucleotide sequence of SEQ ID NO: 23 and a solution of a primer having the nucleotide sequence of SEQ ID NO: 24 prepared into 3 μM, each 2.5 μL of each 2 mM of dNTPs, 2.5 μL of a 10× buffer (100 mM Tris-HCl pH 8.3, 500 mM KCl, 15 mM MgCl₂, 0.01% Gelatin), 0.125 μL of 5 U/μL of thermostable DNA polymerase (AmpliTaq Gold), and 5 μL of a 5 N aqueous betaine solution, and the mixture was added with sterilized ultrapure water to make the liquid volume 25 μL. PCR was conducted in such a condition that the reaction solution was kept at 95° C. for 10 minutes, followed by 35 cycles each including 30 seconds at 95° C., 60 seconds at 63° C. and 45 seconds at 72° C.

After conducting PCR, amplification was examined by 1.5% agarose gel electrophoresis. The result was as follows (these correspond to Third step of the present measuring method).

As for the DNA fragment X2, in the case of Group A (untreated group), amplification was observed, and its amplification product was obtained. On the other hand, in the case of Group B (HpaII, HhaI treated group), amplification was not observed, and its amplification product was not obtained. On the other hand, as for the DNA fragment Y2, amplification was observed and its amplification product was obtained both in the cases of Group A (untreated group) and Group B (HpaII, HhaI treated group).

From the above, it was confirmed that by using a serum solution as a DNA sample derived from a genomic DNA contained in a biological specimen, in the same manner as in Example 1, single-stranded DNA containing the objective DNA region can be selected, and only methylated DNA can be amplified to a detectable level without amplifying unmethylated DNA in the objective DNA region, and an amount of amplified DNA can be quantified.

### Example 3

Using the DNA fragment X2 and the DNA fragment Y2 obtained in Example 1, the following test was conducted.

Solutions containing 0, 0.1, 1 and 10 pg of the DNA fragment X2 and the DNA fragment Y2 in 10 μL of a TE buffer (10 mM Tris, 1 mM EDTA, pH 8.0) were prepared. Each 10 μL of these TE buffer solutions was individually added with 1 μL of 5 μM biotinated oligonucleotide B1, 2 μL of a 10× annealing buffer (330 mM Tris-Acetate pH 7.9, 660 mM KOAc, 100 mM MgOAc2, 5 mM dithiothreitol), and 7 μL of sterilized ultrapure water, to obtain a mixture.

Then the obtained mixture was heated at 95° C. for 5 minutes, and then rapidly cooled to 50° C., and kept at the temperature for 5 minutes. After keeping at 37° C. for 5 minutes, the mixture was returned to room temperature, to cause base-pairing between single-stranded DNA (the DNA fragment X2 or the DNA fragment Y2) containing the objective DNA region and biotinated oligonucleotide (each two samples were prepared for the DNA fragment X2 and the DNA fragment Y2).

Then, the mixture prepared in the manner as describe above was introduced into a PCR tube coated with streptavidin, and kept at 37° C. for 5 minutes, whereby the biotinated oligonucleotide was immobilized on the support coated with streptavidin (these correspond to First step of the present measuring method).

After removing the solution from the PCR tube, 100 μL of a washing buffer (0.05% Tween 20-containing phosphate buffer: 1 mM KH2PO4, 3 mM Na2HPO.7H2O, 154 mM NaCl, pH 7.4) was added, and the washing buffer was removed by pipetting. This operation was executed another two times.

After that, the double-stranded DNA thus obtained was subjected to the following two kinds of treatments.

Group A (untreated group): The double-stranded DNA prepared above was added with 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL.
Group B (HpaII and HhaI digestion treatment group): The double-stranded DNA prepared above was added with each 15 U of HpaII and HhaI, 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL.

After incubating each reaction solution at 37° C. for 5 hours (digestion treatment), the supernatant was removed, 100 μL of the washing buffer was mixed, and the washing buffer was removed by pipetting (these correspond to Second step of the present measuring method).

Then, from the obtained undigested substance, PCR was conducted using the following primers and reaction conditions. If the objective DNA region is methylated, DNA (having the nucleotide sequence of SEQ ID NO: 22, a sequence corresponding to base No. 76669 to 76835 in the GPR7 sequence shown in Genbank Accession No. AC009800 and so on) would be amplified.

| | | |
|---|---|---|
| PF4: | 5′-GACAACGCCTCGTTCTCGG-3′ | (SEQ ID NO: 23) |
| PR4: | 5′-GCGGAGTTGCCCGCCAGA-3′ | (SEQ ID NO: 24) |

As a reaction solution of PCR, the genomic DNA which is a template was mixed with each 0.3 μL of a solution of a primer having the nucleotide sequence of SEQ ID NO: 23 and a solution of a primer having the nucleotide sequence of SEQ ID NO: 24 prepared into 50 μM, each 5 μL of each 2 mM of dNTPs, 5 μl of a 10× buffer (100 mM Tris-HCl pH 8.3, 500 mM KCl, 15 mM MgCl₂, 0.01% Gelatin), 0.25 μl of 5 U/μL of thermostable DNA polymerase (AmpliTaq Gold), and 10 μL of a 5 N aqueous betaine solution, and the mixture was added with sterilized ultrapure water to make the liquid volume 50 μL. PCR was conducted in such a condition that the reaction solution was kept at 95° C. for 10 minutes, followed by 32 cycles each including 30 seconds at 95° C., 30 seconds at 59° C. and 45 seconds at 72° C.

After conducting PCR, amplification was examined by 1.5% agarose gel electrophoresis. The result was as follows (these correspond to Third step of the present measuring method).

As for the DNA fragment X2, in the case of Group A (untreated group), amplification was observed in a sample of 1 pg/10 μL and in a sample of 10 pg/10 μL, and its amplification product was obtained.

On the other hand, in Group B (HpaII, HhaI treated group), its amplification product was not obtained in any sample. On the other hand, as for the DNA fragment Y2, in samples of 1 pg/10 μL and 10 pg/10 μL in Group A (untreated group) and Group B (HpaII, HhaI treated group), amplification was observed, and its amplification product was obtained.

These revealed that by using a solution containing a divalent cation (a magnesium ion) as an annealing buffer and causing base-pairing between single-stranded DNA containing an objective DNA region (plus strand) and single-stranded immobilized oligonucleotide having a nucleotide sequence complementary to the objective DNA region of the single-stranded DNA, it is possible to select single-stranded DNA containing the objective DNA region as is the case with the foregoing Example 1 and Example 2, and by the treatment with the methylation sensitive restriction enzyme, only methylated DNA can be amplified to a detectable level without amplifying unmethylated DNA in the objective DNA region, and an amount of amplified DNA can be quantified.

### Example 4

Using the DNA fragment X2 and the DNA fragment Y2 obtained in Example 1, the following test was conducted.

Solutions containing 0, 0.1, 1, and 10 pg of the DNA fragment X2 and the DNA fragment Y2 in 10 μL of rat serum were prepared. Each 10 μl of these solutions was individually added with 1 μL of 5 μM biotinated oligonucleotide B1, 2 μL of a 10× annealing buffer (330 mM Tris-Acetate pH 7.9, 660 mM KOAc, 100 mM MgOAc2, 5 mM dithiothreitol), and 7 μL of sterilized ultrapure water, to obtain a mixture.

Then, the obtained mixture was heated at 95° C. for 5 minutes. Then, the mixture was rapidly cooled to 50° C., and kept at this temperature for 5 minutes. Then the mixture was kept at 37° C. for 5 minutes, and returned to room temperature, to allow base-pairing between single-stranded DNA (the DNA fragment X2 or the DNA fragment Y2) containing the objective DNA region, and biotinated oligonucleotide (two were prepared for each of the DNA fragment X2 and the DNA fragment Y2).

Then, the mixture prepared in the manner as describe above was introduced into a PCR tube coated with streptavidin, and kept at 37° C. for 5 minutes, whereby the biotinated oligonucleotide was immobilized on the support coated with streptavidin (these correspond to First step of the present measuring method).

Then, after removing the solution from the PCR tube, 100 μL of a washing buffer (0.05% Tween 20-containing phosphate buffer: 1 mM KH2PO4, 3 mM Na2HPO.7H2O, 154 mM NaCl, pH 7.4) was added, and the washing buffer was removed by pipetting. This operation was repeated another two times.

After that, the double-stranded DNA thus obtained was subjected to the following two kinds of treatments.

Group A (untreated group): The double-stranded DNA prepared above was added with 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL.
Group B (HpaII and HhaI digestion treatment group): The double-stranded DNA prepared above was added with each 15 U of HpaII and HhaI, 3 μL of a 10× buffer (330 mM Tris-Acetate pH 7.9, 660 mM KOAc, 100 mM MgOAc2, 5 mM dithiothreitol) which is optimum for HpaII and HhaI, and 3 μL of 10×BSA (Bovine serum albumin 1 mg/mL), and the resultant mixture was added with sterilized ultrapure water to make the liquid volume 30 μL.

After incubating each reaction solution at 37° C. for 5 hours (digestion treatment), the supernatant was removed, 100 μL of the washing buffer was added, and the washing buffer was removed by pipetting (these correspond to Second step of the present measuring method).

Then, from the obtained undigested substance, PCR was conducted using the following primers and reaction conditions. If the objective DNA region is methylated, DNA (having the nucleotide sequence of SEQ ID NO: 22, a sequence corresponding to base No. 76669 to 76835 in the GPR7 sequence shown in Genbank Accession No. AC009800 and so on) would be amplified.

| | | |
|---|---|---|
| PF4: | 5′-GACAACGCCTCGTTCTCGG-3′ | (SEQ ID NO: 23) |
| PR4: | 5′-GCGGAGTTGCCCGCCAGA-3′ | (SEQ ID NO: 24) |

As a reaction solution of PCR, the genomic DNA which is a template was mixed with each 0.3 μL of a solution of a primer having the nucleotide sequence of SEQ ID NO: 23 and a solution of a primer having the nucleotide sequence of SEQ ID NO: 24 prepared into 50 μM, each 5 μL of each 2 mM of dNTPs, 5 μL of a 10× buffer (100 mM Tris-HCl pH 8.3, 500 mM KCl, 15 mM MgCl₂, 0.01% Gelatin), 0.25 μL of 5 U/μL of thermostable DNA polymerase (AmpliTaq Gold), and 10 μL of a 5 N aqueous betaine solution, and the mixture was added with sterilized ultrapure water to make the liquid volume 50 μL. PCR was conducted in such a condition that the reaction solution was kept at 95° C. for 10 minutes, followed by 32 cycles each including 30 seconds at 95° C., 30 seconds at 59° C. and 45 seconds at 72° C.

After conducting PCR, amplification was examined by 1.5% agarose gel electrophoresis. The result was as follows (these correspond to Third step of the present measuring method).

As for the DNA fragment X2, in the case of Group A (untreated group), amplification was observed in a sample of 1 pg/10 μL and in a sample of 10 pg/10 μL, and its amplification product was obtained.

On the other hand, in Group B (HpaII, HhaI treated group), its amplification product was not obtained in any sample. Meanwhile, as for the DNA fragment Y2, in samples of 1 pg/10 μL and 10 pg/10 μL in Group A (untreated group) and Group B (HpaII, HhaI treated group), amplification was observed, and its amplification product was obtained.

These revealed that by using a serum solution as a DNA sample derived from a genomic DNA contained in a biological specimen, and using a solution containing a divalent cation (a magnesium ion) as an annealing buffer and causing base-pairing between single-stranded DNA containing an objective DNA region (plus strand) and single-stranded immobilized oligonucleotide having a nucleotide sequence complementary to the objective DNA region of the single-stranded DNA, it is possible to select single-stranded DNA containing the objective DNA region as is the case with the foregoing Example 3, and by the treatment with the methylation sensitive restriction enzyme, only methylated DNA can be amplified to a detectable level without amplifying unmethylated DNA in the objective DNA region, and an amount of amplified DNA can be quantified.

### Example 5

A mammal-derived breast cancer cell line MCF-7 (ATCC NO. HTB-22) purchased from ATCC was cultured to confluent in a special medium for the cell line described in an ATCC catalogue, to obtain about 1×10⁷ cells. The obtained cells were added with 10-times volume of a SEDTA buffer [10 mM Tris-HCl pH 8.0, 10 mM EDTA pH 8.0, 100 mM NaCl] and homogenized. After adding the obtained mixture with 500 pg/mL of proteinase K (Sigma) and sodium dodecyl sulfate in a concentration of 1% (w/v), the mixture was shaken at 55° C. for about 16 hours. After completion of shaking, the mixture was subjected to extraction with phenol [saturated in 1 M Tris-HCl, pH 8.0)]/chloroform. The aqueous layer was collected, added with NaCl in a concentration of 0.5 N, subjected to ethanol precipitation, and the generated precipitate was collected. The collected precipitate was dissolved in a TE buffer (10 mM Tris, 1 mM EDTA, pH 8.0), added with RNase A (Sigma) in a concentration of 40 μg/mL, and incubated at 37° C. for 1 hour. The incubated mixture was subjected to phenol/chloroform extraction. The aqueous layer was collected, added with NaCl in a concentration of 0.5 N, and subjected to an ethanol precipitation treatment, to collect the generated precipitates (a genomic DNA). By rinsing the collected precipitates with 70% ethanol, the genomic DNA was obtained.

By conducting PCR using the obtained genomic DNA as a template and using the following primers and reaction conditions, a DNA fragment (DNA fragment X1, having the nucleotide sequence of SEQ ID NO: 25, a sequence corresponding to base No. 8 to 480 in a LINE1 sequence as shown in Genbank Accession No. M80343 and so on) containing the nucleotide sequence of SEQ ID NO: 17 (a region corresponding to base No. 257 to 352 in a LINE 1 sequence shown in Genbank Accession No. M80343 and so on) used as a test sample was amplified.

| | | |
|---|---|---|
| PF1: | 5′-GAGCCAAGATGGCCGAATAGG-3′ | (SEQ ID NO: 26) |
| PR1: | 5′-CTGCTTTGTTTACCTAAGCAAGC-3′ | (SEQ ID NO: 27) |

As a reaction solution of PCR, 2 ng of the genomic DNA which is a template, each 0.125 uL of a solution of a primer having the nucleotide sequence of SEQ ID NO: 26 prepared into 100 μmol/μL and a solution of a primer having the nucleotide sequence of SEQ ID NO: 27, each 2.5 μL of 2 mM of dNTPs, 2.5 μL of a 10× buffer (100 mM Tris-HCl pH 8.3, 500 mM KCl, 15 mM MgCl₂, 0.01% Gelatin) and 0.125 μL of 5 U/μL of thermostable DNA polymerase were mixed, and added with sterilized ultrapure water to make the liquid volume 25 μL. After keeping the reaction solution at 95° C. for 10 minutes, a cycle consisting of 30 seconds at 95° C., 60 seconds at 63° C., and 45 seconds at 72° C. was repeated 50 times to conduct PCR.

After conducting PCR, amplification was examined by 1.5% agarose gel electrophoresis, and an objective DNA fragment (473 bp, DNA fragment X1) was cut out, and purified by using QIAGEN QIAquick Gel Extraction Kit (available from QIAGEN).

A part of the obtained DNA fragment X1 was treated with a methylation enzyme SssI (available from NEB), to obtain a DNA fragment (hereinafter, denoted by a DNA fragment Y1) in which every 5-CG-3′ was methylated. Also in this case, likewise the above case, amplification was examined by 1.5% agarose gel electrophoresis, and an objective DNA fragment (473 bp, DNA fragment Y1) was cut out, and purified by using QIAGEN QIAquick Gel Extraction Kit (available from QIAGEN).

Using the DNA fragment X1 and the DNA fragment Y1, the following mixtures of a methylated fragment and an unmethylated fragment were prepared.

TABLE 1

| | Abundance of DNA | Abundance of DNA | |
|---|---|---|---|
| | fragment X1 | fragment Y1 | |
| | containing | containing | |
| DNA | objective DNA | objective DNA | Methylation |
| fragment | region | region | rate |
| I | 100% | 0% | 0% |
| II | 90% | 10% | 10% |
| III | 75% | 25% | 25% |
| IV | 50% | 50% | 50% |
| V | 0% | 100% | 100% |

Using DNA fragments I to V, the following four kinds of solutions were prepared.

Group A (untreated group): About 25 ng of a DNA fragment was added with 2 μL of a 10× buffer (330 mM Tris-Acetate pH 7.9, 660 mM KOAc, 100 mM MgOAc2, 5 mM dithiothreitol) which is optimum for HpaII and HhaI, and 2 μL of 10×BSA (Bovine serum albumin 1 mg/mL), and the resultant mixture was added with sterilized ultrapure water to make the liquid volume 20 μl.
Group B (HpaII treated group): About 25 ng of a DNA fragment was added with 0.5 U of HpaII, 2 μL of a 10× buffer (330 mM Tris-Acetate pH 7.9, 660 mM KOAc, 100 mM MgOAc2, 5 mM dithiothreitol) optimum for HpaII and HhaI, and 2 μL of 10×BSA (Bovine serum albumin 1 mg/mL), and the mixture was then added with sterilized ultrapure water to make the liquid volume 20 μL.
Group C (HhaI treated group): About 25 ng of a DNA fragment was added with 0.5 U of HhaI, 2 μl of a 10× buffer (330 mM Tris-Acetate pH 7.9, 660 mM KOAc, 100 mM MgOAc2, 5 mM dithiothreitol) optimum for HpaII and HhaI, and 2 μL of 10×BSA (Bovine serum albumin 1 mg/mL), and the mixture was then added with sterilized ultrapure water to make the liquid volume 20 μL.
Group D (HpaII and HhaI treated group): About 25 ng of a DNA fragment was added with each 0.5 U of HpaII and HhaI, 2 μL of a 10× buffer (330 mM Tris-Acetate pH 7.9, 660 mM KOAc, 100 mM MgOAc2, 5 mM dithiothreitol) optimum for HpaII and HhaI, and 2 μL of 10×BSA (Bovine serum albumin 1 mg/mL), and the mixture was then added with sterilized ultrapure water to make the liquid volume 20 μL.

After incubating each reaction solution at 37° C. for 2 hours, the solution was ×100 diluted by adding sterilized ultrapure water.

Using 5 μL of each diluted solution (an amount corresponding to 62.5 pg of the DNA fragment) as a template, real-time PCR was conducted using the following primers PF2 and PR2 and probe T1 in which the 5′-end is labeled with a reporter fluorescent pigment FAM (6-carboxy-fluorescein) and the 3′-end is labeled with a quencher fluorescent pigment TAMRA (6-carboxy-tetramethyl-rhodamine), in order to determine a DNA amount in the region having the nucleotide sequence of SEQ ID NO: 17.

| | |
|---|---|
| <Primers> | |
| PF2 (forward side): | |
| 5′-CACCTGGAAAATCGGGTCACT-3′ | (SEQ ID NO: 28) |
| PR2 (reverse side): | |
| 5′-CGAGCCAGGTGTGGGATATA-3′ | (SEQ ID NO: 29) |
| <Probe> | |
| T1: | |
| 5′-CGAATATTGCGCTTTTCAGACCGGCTT-3′ | (SEQ ID NO: 30) |

As a reaction solution of PCR, 62.5 pg of the DNA fragment which is a template, each 2.5 μL of a solution of a primer having the nucleotide sequence of SEQ ID NO: 28 and a solution of a primer having the nucleotide sequence of SEQ ID NO: 29 prepared to 3 μmol/μL, 2.5 μL of a probe having the nucleotide sequence of SEQ ID NO: 30 prepared to 2.5 μmol/μL, each 2.5 μL of 2 mM of dNTPs, 2.5 μL of a 10×PCR buffer (100 mM Tris-HCl pH 8.3, 500 mM KCl, 15 mM MgCl₂, 0.01% Gelatin), and 0.125 μL of 5 U/μL of thermostable DNA polymerase (AmpliTaq Gold) were mixed, and added with sterilized ultrapure water to make the liquid volume 25 μL. Real-time PCR was conducted using Gene Amp 5700 Sequence Detection System (Applied Biosystems). For amplifying the region (DNA) having a nucleotide sequence represented by base No. 1 to 94 in the nucleotide sequence of SEQ ID NO: 17, after keeping the reaction solution at 95° C. for 10 minutes, real-time PCR was conducted with 15 seconds at 95° C. and 60 seconds at 60° C. being one cycle. From the result of real-time PCR, the DNA amount of the region was quantified. Tests were conducted three times for each biological specimen.

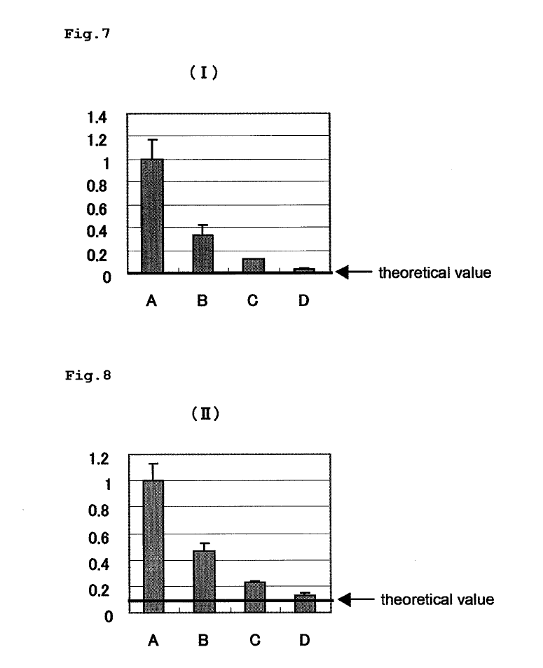

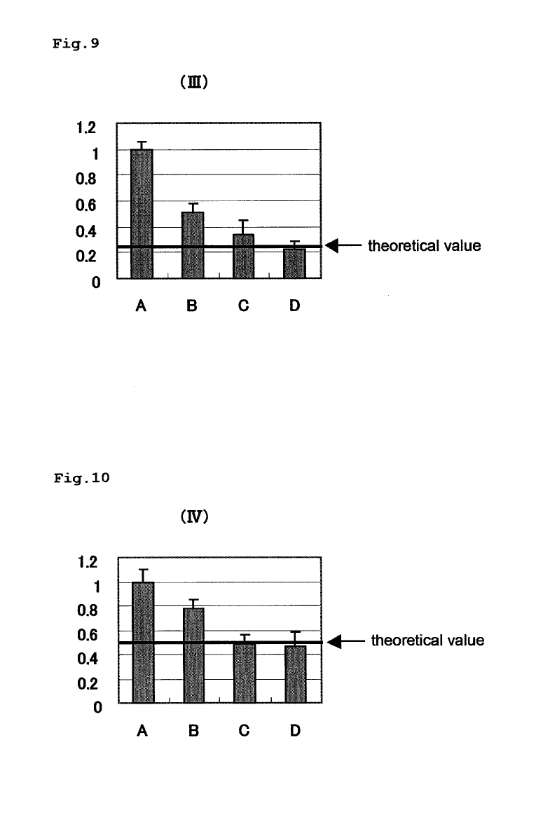

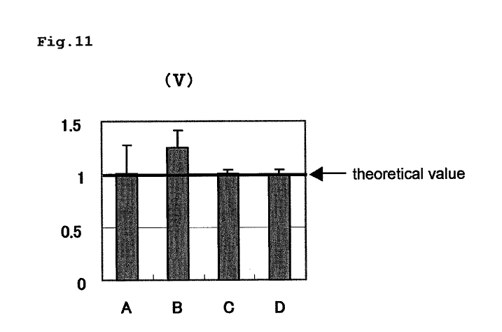

The results are shown in FIGS. 7 to 11. Assuming the DNA amount of the region in Group A as 1, the DNA amounts in the region in other groups are shown. Since FIG. 7 (“I”) is a mixture of a fragment having a methylation rate of 0%, a theoretical value in Group B, Group C and Group D is “0”; since FIG. 8 (“II”) is a fragment having a methylation rate of 10%, a theoretical value in Group B, Group C and Group D is “0.1”; since FIG. 9 (“III”) is a fragment having a methylation rate of 25%, a theoretical value in Group B, Group C and Group D is “0.25”; since FIG. 10 (“IV”) is a fragment having a methylation rate of 50%, a theoretical value in Group B, Group C and Group D is “0.5”; since FIG. 11 (“V”) is a fragment having a methylation rate of 100%, a theoretical value in Group B, Group C and Group D is “1”. As a result of the test, as shown in FIGS. 7 to 11, a value closest to the theoretical value is obtained in Group D, and it was revealed that a digestion treatment with two or more kinds of methylation-sensitive enzymes is preferred.

### INDUSTRIAL APPLICABILITY

Based on the present invention, it becomes possible to provide a method of measuring the content of methylated DNA in an objective DNA region in a genomic DNA contained in a biological specimen in a simple and convenient manner, and so on.

### Free Text in Sequence Listing

### SEQ ID NO:19

Designed oligonucleotide primer for PCR

### SEQ ID NO:20

Designed oligonucleotide primer for PCR

### SEQ ID NO:21

Designed biotinated oligonucleotide for fixation

### SEQ ID NO:23

Designed oligonucleotide primer for PCR

### SEQ ID NO:24

Designed oligonucleotide primer for PCR

### SEQ ID NO:26

Designed oligonucleotide primer for PCR

### SEQ ID NO:27

Designed oligonucleotide primer for PCR

### SEQ ID NO:28

Designed oligonucleotide primer for PCR

### SEQ ID NO:29

Designed oligonucleotide primer for PCR

### SEQ ID NO:30

Designed oligonucleotide probe for Real-Time PCR

1. A method of measuring the content of methylated DNA in an objective DNA region in a genomic DNA contained in a biological specimen, comprising:
(1) First step of causing base-pairing between a single-stranded DNA (plus strand) containing an objective DNA region and a single-stranded immobilized oligonucleotide having a nucleotide sequence that is complementary to the objective DNA region of the single-stranded DNA, and selecting the single-stranded DNA from a DNA sample derived from a genomic DNA contained in a biological specimen, and forming double-stranded DNA made up of the selected single-stranded DNA and the single-stranded immobilized oligonucleotide that are base-paired;
(2) Second step of digesting the double-stranded DNA formed in First step with one or more kind of methylation-sensitive restriction enzyme, and removing generated free digests (double-stranded DNA containing one or more unmethylated CpG pair in a recognition site of the methylation-sensitive restriction enzyme); and
(3) Third step comprising, as pre steps of each of the following regular steps:
Step (First pre step) of temporarily separating the formed double-stranded DNA (formed double-stranded DNA not containing an unmethylated CpG pair in a recognition site of the methylation-sensitive restriction enzyme) which is an undigested substance obtained in Second step, in a single-stranded state;
Step (Second pre step) comprising:
Step (Second (A) pre step) of causing base-pairing between the generated free single-stranded DNA (plus strand) and the single-stranded immobilized oligonucleotide, and selecting the generated free single-stranded DNA, and forming double-stranded DNA made up of the selected single-stranded DNA and the single-stranded immobilized oligonucleotide that are base-paired, and
Step (Second (B) pre step) of making the double-stranded DNA formed in Step (Second (A) pre step) into double-stranded DNA extended and formed from the selected single-stranded DNA by extending a primer once using the selected single-stranded DNA as a template and the single-stranded immobilized oligonucleotide as a primer; and
Step (Third pre step) of temporarily separating the double-stranded DNA (extensionally-formed double-stranded DNA not containing an unmethylated CpG pair in a recognition site of the methylation-sensitive restriction enzyme) extended and formed in Second pre step into a single-stranded DNA (plus strand) and a single-stranded DNA (minus strand), and as regular steps, (a) Step A (a regular step) comprising:
Step A1 of causing base-paring between the generated single-stranded DNA (plus strand) and the single-stranded immobilized oligonucleotide (minus strand), and selecting the single-stranded DNA, and
Step A2 of extending and forming double-stranded DNA from the single-stranded DNA by one extension of a primer using the single-stranded DNA selected in Step A1 as a template and the single-stranded immobilized oligonucleotide as a primer,
(b) Step B (a regular step) of making the single-stranded DNA into extensionally-formed double-stranded DNA by one extension of oligonucleotide by using the single-stranded generated DNA (minus strand) as a template, and oligonucleotide (a reverse primer) having a nucleotide sequence (plus strand) complementary to a partial nucleotide sequence (minus strand) of a nucleotide sequence possessed by the DNA in a single-stranded state (minus strand), wherein the partial nucleotide sequence (minus strand) is positioned on the 3′-end side than the 3′-end of the nucleotide sequence (minus strand) complementary to the nucleotide sequence (plus strand) of the objective DNA region, and is not usable in an extension reaction using the single-stranded immobilized oligonucleotide as a template, as an extension primer,
wherein the methylated DNA in the objective DNA region is amplified to a detectable level by repeating each regular step of Third step after temporarily separating the extensionally-formed double-stranded DNA obtained in each regular step into a single-stranded state, and the amplified DNA is quantified.

2. The method according to claim 1, wherein in First step, base pairing is conducted in a reaction system containing a divalent cation when the single-stranded DNA (plus strand) containing the objective DNA region and the single-stranded immobilized oligonucleotide having a nucleotide sequence complementary to the objective DNA region of the single-stranded DNA are base-paired.

3. The method according to claim 2, wherein the divalent cation is a magnesium ion.

4. The method according to claim 1, further comprising in a previous operation stage of First pre step in Third step:
a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and
additionally comprising the following one step as a respective regular step of Third step:
(c) Step C (a regular step) comprising:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

5. The method according claim 1, further comprising in a post operation stage of First pre step in Third step:
a step (Additional pre step) of adding single-stranded oligonucleotide (minus strand) in a free state having a nucleotide sequence complementary to a part of the 3′-end of single-stranded DNA (plus strand) containing the objective DNA region into a reaction system, and
a step (additional re-pre step) of temporarily separating into a single-stranded state double-stranded DNA (extensionally-formed double-stranded DNA not containing an unmethylated CpG pair in the recognition site of the methylation-sensitive restriction enzyme) which is an undigested substance obtained through Second step and Additional pre step, and
additionally comprising the following one step as a respective regular step of Third step:
(c) Step C (a regular step) comprising:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

6. The method according to claim 1, further comprising in a previous operation stage of Third pre step in Third step:
a step (Additional pre step) of adding single-stranded oligonucleotide (minus strand) in a free state having a nucleotide sequence complementary to a part of the 3′-end of single-stranded DNA (plus strand) containing the objective DNA region into a reaction system, and
additionally comprising the following one step as a respective regular step of Third step:
(c) Step C (a regular step) comprising:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

7. The method according to claim 1, further comprising in a post operation stage of Third pre step in Third step:
a step (Additional pre step) of adding single-stranded oligonucleotide (minus strand) in a free state having a nucleotide sequence complementary to a part of the 3′-end of single-stranded DNA (plus strand) containing the objective DNA region into the reaction system, and
a step (additional re-pre step) of temporarily separating into a single-stranded state double-stranded DNA (extensionally-formed double-stranded DNA not containing an unmethylated CpG pair in the recognition site of the methylation sensitive restriction enzyme) which is an undigested substance obtained through Second step and Additional pre step, and
additionally comprising the following one step as a respective regular step of Third step:
(c) Step C (a regular step) comprising:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by one extension of a primer by using the single-stranded DNA selected in Step C1 as a template and the single-stranded oligonucleotide (minus strand) as a primer.

8. A method of measuring a methylation rate additionally comprising the following two steps as steps of the method as described in claim 1:
(4) Fourth step of amplifying DNA (total amount of methylated DNA and unmethylated DNA) of the objective DNA region to a detectable level by conducting Third step after conducting First step without conducting Second step, and quantifying the amplified DNA; and
(5) Fifth step of calculating a rate of methylated DNA in the objective DNA region based on a difference obtained by comparing the DNA amount quantified by Third step, and the DNA amount quantified in Fourth step.

9. The method according to claim 1, wherein the biological specimen is mammalian serum or plasma.

10. The method according to claim 1, wherein the biological specimen is mammalian blood or bodily secretion.

11. The method according to claim 1, wherein the biological specimen is a cell lysate or a tissue lysate.

12. The method according to claim 1, wherein the DNA sample derived from a genomic DNA contained in a biological specimen is a DNA sample digested in advance with a restriction enzyme whose recognition cleavage site excludes the objective DNA region possessed by the a genomic DNA.

13. The method according to claim 1, wherein the DNA sample derived from a genomic DNA contained in a biological specimen is a DNA sample digested with one or more kind of methylation-sensitive restriction enzyme.

14. The method according to claim 1, wherein the DNA sample derived from a genomic DNA contained in a biological specimen is a DNA sample purified in advance.

15. The method according to claim 1, wherein the one or more kind of methylation-sensitive restriction enzyme is a restriction enzyme having its recognition cleavage site in the objective DNA region possessed by a genomic DNA contained in the biological specimen.

16. The method according to claim 1, wherein the one or more kind of methylation-sensitive restriction enzyme is HpaII or HhaI which is a methylation sensitive restriction enzyme.


SEQUENCE LISTING (30 sequences)

SEQ ID NO: 1 - DNA, 2661 bp, Homo sapiens
acagacatgtgccaccatgcccagctaattttttgtttgtttgtttgtttgtttgtatttttagtagagatggggttttgccatgttggccaggctggtctcgaactcctgacctcgaatgataatgatccgccgcttggcctccaaagtgctaggattacaggtgtgagccactgcgccaggcctgggcactttctttagtagtttgaggagcaacatttttgacagtgtccttctgctcaagattcaagatcccagataaaattaaaccatctagagagatggcttgattggccaaacctggatctcatgaccacttcttgaagtgggtaagtctcataaatgctcagtccttccactatgcaactgagtggggtgggtgggaagcccctcaaaggaaaatccggttgttcttactagaaagaaaaggaaaatggatgtgaggcagtcaaaatcagcagaggtccaccacaccaccaaaatgtggtgattaaatatggagagacagagactaacagaggtatgtgaatattgaagtatgtctggacaatagcccaatgatgagaccaataaaatggttaccaaaatctggttttgagtagtagtgttaaatcagaccatttagtaaccattttttgttgcaaagtttctagcactgcccaaaccctgagtggtatatgaataactcgtccattatgtatctctttccagtcagcataatttatcccccacctatattcttttctgaccactcctacttccttctctttaccaaaatctaaactctaaggctgtttcttcagcaacttctttgtttagattggaagataaattaaacagcatgcgatgttttactgactttcagtatttaacagaggtgatttaatttttttttaaatccaaagtcaaacttctttataagatgaaggagaaaaatgtcttataaaatgcatatgtgaagatgccttctgagtgctttctcatgcagacttgttctagtctttaatgaatcttccttgtagacactgtggagatgaaagatggttctccacttctactcaaagtacaaatcaggccggcattttgaaaaagagacaggtttattcatagctgcagcgttagctggctttgttccctgtacaatttcacttttggttattaaaatattcactgtaggaaataaatttgtaacccatttctcatattacctacacacagaaaaacaaaatttgatatcctggggtttatttgctgagggcgcttcccataaaagcgagagagtgtgcgttgggaaatgtgtctggttaactcttttatggataaactttagtcacaatcctcccccgcccccctctcacccccagcaccctcccaacctcccgacttcccgcctctcaagggctggtgacctaatagcatttttcttcgtgcatattttggcgtcgccccatggcctggctgccttcgcctgtctgagttttttgaaattcctgcatgttcgccccagattaagccagtgtgtctcaggatgtgtgttccgttttgttctttccccttaacgctccctgtgcaacgtgtctggggggaggagggcagggacgggagagagggaggggcagaggcgaggagctgtccgccttgcacgtttccaatcgcattacgtgaacaaatagctgaggggcggccgggccagaacggcttgtgtaactttgcaaacgtgccagaaagtttaaatctctcctccttccttcactccagacactgcccgctctccgggactgccgcgcggctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacg

SEQ ID NO: 2 - DNA, 1953 bp, Homo sapiens
tataaattccacgcaggcattgaattgaatttgttcttaaccaaatgcgttttatctatacctggcaggaatctagaagtgaaattacaagatttatttcattttaattctattatgaagcatttaatcacaaataccctgaaaatgaaaagataatttatcattttaccttgactgagcaactctcctcacttcacattcatgaatccataacgcagagaggagactggatgattaagtgtttgattagagaaaacagattaacctagcaaacataataaatttggctcataagcaggatggctttataaatgctcacaatacctctcctgtataaaatcatgaaccacttcctacagtgatgactccatcgaaatagttgagaaacataaagcaaatgcatgtttatggctttctctttgagacattaaaagggtattgaaaggcatatctgattcagcttataactctggatatatattaaggaacatgtaagaaaatattaatgcataaaaaaagctacaacttctcaagtgttctagtttccactttgtcaataattacgttttcaatgtccttctgtggactgtttccaaaggtgccaatccagacccaaagtttcagatcactcagattcacccttaaccttcataacacaacccaatagctttacgaaaaaagttgcatatttaggtagttgttatcccattatgacaaaatacataaaattagcgagatattttttagccttcaaataagtgggaaaaaatccttttagctgagattccatttacatcagaataaaaatctaagttatgactaggttgaagcaacgtcctgtgcagcgctccataaagttcacttagtcttcaagggttccttacttagctaggttagtattcctggcctctttttttagcagtgagaaaaaggatactctccctgccccagctttatttttaaactcacagccatatcctggaggtctctgctggctatttggcgcgtgggggcggaggggggccgggggaggggggcggggcggggtctggaggtctgtgctggctatctggcgtgtgtgtgtgtgtgtgtgtgtgtgtgtgtggttggaggtctctgctggctatctggcgtgtgtgtgtgtgtggtgtggtgtgtgtaagcagtgaggttgttttagggccagtccttcctccgccactttgctgactcaaagacccagaggctttcttggggtgcaggtaccatgattccttgggccctaagggaatttttgttaggctagaagagtgggtgtactcatgatgggtgtacccgaacattcctgggctcaacaaaaccgattatctttataaccgcggcgcctagcacagcgcctggtgccctaaacgttggctgcgggaacgtccgagacgcgggtgcggagccgggggcggaataactggttgcgcggcgctttgaccgtaggcgctggagcgcgtgcgttgcgtgcgcgcgcggaggcggctgcgtcggggcgcgagaaggtgcagttccccggcgggcgggcgggcgggcgggcgaagctgggctcggggccaagcgaggtctagccggagcgactgtgccccgcctcctgggcggagcgggcggctccccatggtcagagcctcgtgccggctcggcagcgcccggacgccgagcccagcgcgtcggccccccggcgtgcgggcgtctcagagccgcggaggggccgccgggaccgtttcagcgtggcggcgctggtgctggcgttggccctggaggacggccccgagtgatggctggcgcctgcctcccgggtgtctcccgggtacagatggagtcgtcccgcggccgccggcggcaaggtcggcagctgcgaggccaagagagaccccaggacacacacagctgcctcccggtgcgagaagaagaccccggcttgagagtgag

SEQ ID NO: 3 - DNA, 889 bp, Homo sapiens
cggccccatggctccgtgtcgtgtccaagggatgggctggcacctcttggaccaggcttaccaccagggcccttctctgaagccccagtctgaccggcctgctgctgggaatccccctctgcccccacactaacctctgctggggctgagccagggcgcgtcggacagtcagggcgacccagccagggcgaccgttggccccgctcctatggggcagcagggaccgacgtcagcagggtggggcgggcacccgagtggtatgccccgccctgccccgcctgcccgccctggtggccgtctgggggcgacaagtcctgagagaaccagacggaagcgcgctgggactgacacgtggacttgggcggtgctgcccgggtgggtcagcctgggctgggaggcagccccgggacacagctgtgcccacgccgtctgagcaccccaagcccgatgcagccacccccagacgaggcccgcagggacatggccggggacacccagtggtccaggtgtggcgggggtgaggggagggggggtgggagcggtggagatggggccgtggggagggagctgagatactgccacgtgggacgatgctaggtggggagggctgagctgggcgggctcctctggctgtggggccccctgtgttccttgtgggaggtggaaggaagtgagtgccctgtccttcctccctgccatgagattccaggaccggacctggcaagtgccctatcccagccagtgttcctggggctcttccaggcagggctatgttccccaggccaggggcattgtcctggacagtcaggaggcatacccctcgccaggtggaaccaccctgtgtatgcatgaccctgacaagcaggcgccaggacagtcaggaggccag

SEQ ID NO: 4 - DNA, 863 bp, Homo sapiens
gttgttgggtgtgaatggagaactgtgggccctccccgacaccttccagcgggacggcaacgggggcccagggggtgggcgccatcaaccccgtcccaccgccaggacggcgcgggggagggccggcgggggcggggcgtcctgtaaggcgcggcccccacccgcgggcggggcggcattcctgggaggccggcgctctgacgtggacccgggggccgcgggcacggcgggggggcggcggtccgggggcttcttaaaccccccgccccggcccagcccgcacttcccgagcaccgctccgaccctggagggagagagagccagagagcggccgagcgcctaggaggcccgccgagcctcgccgagccccgccagccccggcgcgagagaagttggagaggagagcagcgcagcgcagcgagtcccgtggtcgcgccccaacagcgcccgacagcccccgatagcccaaaccgcggccctagccccggccgcacccccagcccgcgccagcatgatgaacaacagcggctactcagacgccggcctcggcctgggcgatgagacagacgagatgccgtccacggagaaggacctggcggaggacgcgccgtggaagaagatccagcagaacacattcacgcgctggtgcaatgagcacctcaagtgcgtgggcaagcgcctgaccgacctgcagcgcgacctcagcgacgggctccggctcatcgcgctgctcgaggtgctcagccagaagcgcatgtaccgcaagttccatccgcgccccaacttccgccaaatgaagctggagaacgtgtccgtggccctcgagttcctcgagcgcgagcacatcaagctcgtgtccatag

SEQ ID NO: 5 - DNA, 2198 bp, Homo sapiens
aagagaggcacactccctctaccacaccgagggagggggcgttgagctgagaaaggttgagagaatgagggacccaggtaggtggacatcggccaagaaaggaaccacagcgggaggtaagaccgagagtccccagcttgaagcgtcaccactccgggattcccagattccaacgcgagcctggggaaagcccacagtggagagagtccggctggcagggaatggccctacccccggggtgaaatctcggagggtcgtgcagccgagtcgcgcctctgcgctgatgcgtgagagatgccggacgtcgcgtttgcctgtgcgagcctcgcggatgctgtgcagtcttggtcccctctgcgtgtgtctaacgccgaatgctggtgtctcgaggtgtgagcttcggggccggtgtctttaaagaaccaaagattcttaaggagtgatgatctgggtagagcggcccgacgtagccgcgctcccaggtctcggtgcgagtcctgcggacagaccagaggagacctgctggccagatgccccgggcccaaggcggacgccagactgtctctgcgccagccgggctggccttcggaatggatcaggcacccgggaggccggagtggatctcagaccctcaagccgggaacaaacccgtcgatgcccgtgggcctggagtccgcctcctccttcccgccccacccctacccctgcctccgaaaggcttcttcgctggtcagtagctgcgtgcccgtctgcctgaggctgggtcagaattggcgggctggtaacgaccccgtgcacaagcggctcccagtctctccagaaagggccgatgactaaggggtgggggtgggggcggagggctggaaggtgttagggaagaacgttagcggcctatcctgtcttcagcagcgccctctcatcttctagctctgacgccgagcagagcagttggagctcgggactgggaactgctggaattcctatttagacttctagacagtctagaaacaagaacctttctttccctgggcctcagtttccttgtctgtaaaatcaaaaggcgggctctaggtgtaggccttcttttcgcttggtgattctggattcctttccttggatccgtggggagggggtggcagcaacagtccagggcgttggccgtcctgtgcctcaagtacgtagtccccgtgcccgccccctcaacacccccagcagcccgcccccctaagcccgcagagcagggagctgagtgggaggggcagaggcggggccggttcccagtccctgctggcggactagagtggcgcgggctgagcgtaaaacctgggatagccactcccccttttccttatccccgcccccctgccattggctcccgggagaggttgacatcaaagccgcggtcttatataagccagatccgcaggggagtccgcagaagggttaaacaggtctttgggcttcggcgacctcgcccgcggcagaaaccggtaagaagacagtgggctgcgcgtctcattttcagccttgcccggactctcccaaagccggcgcccagtagtggctccagagcccacaggtggcccccggcagtctctggggcgcatggagcggcgttaatagggctggcggcgcaggccagtagccgctccaacatgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctg

SEQ ID NO: 6 - DNA, 1945 bp, Homo sapiens
ctggatgacagagtgagactccgtctcaaaaaaaaagctccatttgggaggccgaggagggtggattacctgaggtcaggagtttgagaccagcctggcccacatagggaaaccccatctctactaaaaatacaaaaattagtcaggtgtggtggctgacacctataatcccagctacttgggaagctgaggcagggagaatcacttgaaccggggaggtggaggttgcagtgagctgagatcatgccactgcactccagcctgggcgacagggtgagattctgtctcaaacaaacaaatttaaaagctccgaatcctccaaaaataccaagattttcctgtcggtaactagagatgggtactgatgattatttttaataggtgattttcaaagatgtgaacgttatccatggagatttaagtctccaaaaggaaaaaaaatgcatacctttatactaaaacttcatcaccagtcaaatttggatcatcactaaattggcttctacacctctctcctaatataaggtacttgtgtaagtttgcagttgtgagacacttatttcctcatttttaatgtcttctcagtagggccactgatatagtcactatttgactgaccagaatggttggcactggtgattggctcataaagtgccctcgatttagggggctcaattatcaaaggtttaaatcctagcccaaaccattgctgtgatgggggttaatcaatgaaccactcagcttcacttgcaaaagcgggatcacaatagccgctttcgtcatgacccagcctaggtgagatttagtacttaagtacactgccaggcacacaaggttaatttaacaatttaacacatttgtttcctcatccatttctccaaaccttccaactaatcctaacgttcgttcggccaaatgggccaggaattcacttaaacaaaaacaaaaaacaaaacaaacaaaaaaacactccctggggcttggggaaggaggcaccgccgcccatgtcgcagtctgggggtggctcagtcctcagcacccagatctacggccataatgctcttcgaggccaaggagcccggatgcggggcgttgccgaaggcgtcttgctcaggctgcgggaaaggagaggggtgggagcggggtgggggcatcgcgacccagggcaaggcggcgagtcgccgtcttcgagtcccacctgtccgaagcggggtgagaaaaggcaaaacatggcaaagccatgcacctcccagggtgggcaactcacggccggtgaacgccggacccttagcagtttccagacctttggaaccggaagcggagcctgagagcgcgcccgagagggcgtgaacgggaccgctttcccggaagtgcttgcggcctctgcccagcgagctgccccggggtctctctggtttcctaatcagggcaacgccgcgggagagaacctttaccttggctgcactaagttctcggtgccactccctggcagggcgggaccttgtttaggccctgtgatcgcgcggttcgtagtagcgcaaggcgcagagtggaccttgacccgcctagggcgggaagagtttggcccgccgggtcccaaagggcagaatggacgggctcctaaatcccagggaatcctctaaattcattgcagaaaacagtcgggatgtgtttattgacagcggaggcgtacggagggtggcagagctgctgctggccaaggcggcggggccagagctgcgcgtggaggggtggaaagcccttcatgagctgaaccccagggcggccgacgaggccgcggtcaactgggtgttcgtgacagacacgctcaacttctccttttggtcggagcaggacgagcacaagtgtgtggtgaggtacagagggaaaacatacagtgggtactggtccctgtgcgccgccgtcaacagagccctcgacgaag

SEQ ID NO: 7 - DNA, 2379 bp, Homo sapiens
aagcttgtggtttacttggacctctgcctcatctttcttcttttgcgcttcagcctgcgcattcgcttcctccactaggctctcatggtgcagaggtttccaagaagatggtgtgaaggccgagatcatttggttatattataaaatagaatgcaaattcacacaagtttttgttttttatttatttatttttttagagatgaggtcttgctatgttgtttagtctggtctcgaactcctggcctcgtgatcctcccaccttgacctcccaaagtgctgggattacaggcctgaggcctgagccactacacccaactgaattcacatttttttttttcttttctgagacggagtctcactctgtcacccagtatggagtgcagtggcgcgactgcggctcactgcaagctccgtctctcgggttcaagtgattctcatgcctcagccccccaagtagctggaattacaggggtgcactaccacacctggctaatttttctgttttagtagagatggggtttcaccatgttgcctggtctcaaactcctgactttaagtgatccacacacctcagcctcccaaagtgctgggattacaggtgtgagcctccacacccggccgaattcacatgaattttaaagtgatgtcttcaaagtggtttcactgtggggatgggcagctttttgttatacatctagaacgttcctcttctgtttctatgaatactcggttggaaagggctgaaaaacggtcttaagagattatctgattcgtttcccagttttattactcacatatcagctgtaatttgagcacgttttctgattgagacaagactcagatggtattaaacattactacaacacatccgggcacggtggctcacgcctgtaatcccagcactttgggaggccgaggcgggcggatcacgaggtcaggagatcgagaccatcctggctaacacggtgaagccctgtctctactaaaaatacaaaaaattaggcgggcatggtggcgggcgcctgtagtcccagctactcgggaggctgaggcaggagaatggcgtgaacccgggaggcggagcttgcagtgagccgagatcgcgccactgcactccagcctgggcgacagagcaagactccatctcaaaaaaaaaaaaaaaaaaaaaaaactacaacactataaattcatatctattataatagtactttgtgcagggccctaccctaagtccttaaccgaacccggaagcgagaagatgacttttgtttgtttttagagatgggcgcctggctctgtcgccagcctggagtgtgggggcgcgatctcgactcacagcagcctccacctcccgagttcaggcgatcttcctgcctcagcccctcgaggagctgggaccaccggcgcgctccatcgcgcccggctaggagctgactttgaatccgggctctgcgcctggccttctgcatctctataagggaagacatctgtgacctcggggcaaaggtcaaattagatcctgggtaggatcctgttcccgctgcccctcgggctggcactgccaggagtactcagagctcaaagctgggatctgcagtcccttacccactcagtgcacgccgcctaaggctttgcgcttcacctttactcacctcgaagccctggacatccgcatctgccctaagacttctcacctcagtagcagaaggaagtcgcgtcagctggccacagcctctctcctaggagaccgtccgggaaaagcgagtcagggtagaccctgaggcccctcagctccggctattttcagatctgtcgctccttcaccctcagcctttcaaacaggccactccaaaaaaaagcccaatcacagccttccttcttctcctggccttccggcactgtccaatcaacgtacgccatctatcggttagtggtgttgcggggccacccttcccgctggtttccctcgtggtgtgtaaaggcagagaggaaaggcgaggggtgttgacgccaggaaggttccatcttggttaagggcaggagtcccttacggacttgtctgaggaaagacaggaaagcgccagcatctccaccttccccggaagcctccctttgccaggcagaaagggtttcccatggggccgcccctggcgccgcgcccggcccacgtacccggggaggccgggccccggaggacgagggaaagcaggccgggcgccgtgagcttcgcggacgtggccgtgtacttctctcccgaggagtgggaatgcctgcggccagcgcagagggccctgtaccgggacgtgatgcgggagaccttcggccacctgggcgcgctgggtgaggccgggccctccggccgggacccccagtccgtcg

SEQ ID NO: 8 - DNA, 933 bp, Homo sapiens
gagacgtactctggctctgtcgcccaggctggagcgcaatggcgccatctcggcgcactgcaacctccacctcccgggttcaagcgattctactgcctcagcctcccgagtagctgggactacaggcgcgcactaccaagcccggctaatttcttttgtatttttagtagagactgggtttcacgatgttggccgggctggtctggaagtcttgacctcaagcgtgcgccctctccgccactgggtaaggcggggggcggaatagggggcttgcaatttcacactagaggcgggcgccgtgggggaaagaagagtcacgtctcccacggttcgtagaggaaggcctgcctgagcctggagcgggggcgggagagccacagtttggcatccccagggcatcccccagcccgcagactaccaggcctccagaggacaggaccccacccccggccacaggccctgcccccagcactccccgcaccccgcctccaagactcctccgcccactccgcacccaacttataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcggctcggccaggggctgcgcgaacacgcggagcgcacccgcagccagctgagcgcgctggagcggcgcc

SEQ ID NO: 9 - DNA, 6096 bp, Homo sapiens
atctgcacctcctcatatagggttgatccaagtttcacagacatcactgagttcttagtggactcagctattggggctgttctcacactttttttttctttgcaagaatcagcaatgggtgcaagtggacctgtgtaggacgtccagtgaaacattgtgttggtgaatcagctagaatccatccaagaactcagccagcctggtgtggggtgagatctgatccttgaatgtccctcagtggcttttagggctggcaggttcagaagggccctctcatcacccccccagggcctcattccttgtttaacactttgctatcacagtcttgaatccttgtaattgaacaatggaccccacattttcactttgcactggtttctgattctgtaaccgatcctgtccccctctcttgtctcattcactctgggaattgtccccacattctgagacctttcagcagtgccccaacgaggttcctgcccttatctgaagctccaccctcacccccatggcggcaccgcaggcagccctgcttttgcgtcccgcgtaggcaggctgtgcaccggagtcacgaccccctgattcagcctaggcagccacagcttgactgctcccgccggacaagccctactgtgctatctgccgctcttcccttcctcttcccagggggtccgcgtcaggggaggcgcagctgtgtgcattccgggagcttcagacccccgtgtccagcagctccttcgtttcctgggtgctggggcggccttcccagcgaagagctcaactcagcgggacgtttggaggctctctgccccaaggcgctggggagtgtgcggcgggacagtcgtgcttgcctttttcactttcagagtgtccacgccccacccgtttggtcactgcaggtcagtccagtccagcccggcccaccccaccggtgcgtgtctgtcgcacgtggcagacgccatactctctgttcttgtttaaagcccaggatctactgggccctggaggcaagaggtgaacgcagcggaatccacgctgagctgcccgggaacggagcttccaaccccagaaggaggactctgtgctcctacaccttaaccctttttagcccgaaacttctccaacttccttggctttgtttagagctcgacagcgccgccccctggcgctcgttgtgaggacagtagaggagagaggcaagggtgtttttaaacagtttgcctctcaccattatgggggcgacccgagggggagacccactcttccgcattcccggtaagtgaaccaccggaagaggtcgaaagtgacggattcccatgtcctcctccagccccccccccaccctgcccatccacaggacggtggctcttcagtgccctttgccgagcaagtggcgtttctatgcacgtgggtatcaattcggactctggacgaaatggaaacctccttagccgacccgggtgggatcagctgggatcctgcgcgctcccctggggggttgccagccactctgttggggtgcaagaagcaccatccttcggaagctgggccgaaactggccaggctgactcgctcccacgcgcccgcccctacccggcgccgcagcaattcacctgccaccgcctctgagccgggtccggacttcggcgccctgacagtgtccccgcgacttccccacccgatgagatggggtctggcgttggccagtgcgtgtccagggactcgcgggtccctggccagccatggggcagagggcgctggtgttaggccagtcttccccaccctgccccgtcaccccagccacacccactgtcctgtgaggccaagcgcgctccgctggtttcctgagccaggcaccttggccgcggacaggatccagctgtctctccttgcgatcctgtcttcggggaagtccacgtcctaggcaggtcctcccaaagtgcccttggtgccgatcacccctcccagcgtcttgcaggtcctgtgcaccacctcccccactccccattcaaagccctcttctctgaagtctccggttcccagagctcttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtg

SEQ ID NO: 10 - DNA, 2500 bp, Homo sapiens
acccacttctgtgtgtggatagtatcctgcaggagagatgttgtctgcagtgtgagctgggcccaccggagtgtgtgaataggatcctgcaggagaaatggaatccggagtgtgagctgcatccgctgtagagggtggataaaatcctgcaggaaagatggcatctggaatgtcagcgggagccaccgacctctgaggatgcaccccgcaggtgtgatgcggggccagttccaaggctgggttaggttttaccctggcttctgtgttgtactctcattctcttcctctttcttctaatacctgctctgggaggcatcaggccatgtccagtgtgcaggccatggagacccacacggcaaggaactggaaccccctgccagcagcctcgggggtccagtccttagatggtgccctgtggtcagcaatgcacctgtgacctccgggctatgtctcgtggtagttgcttttgtgttttaacatagcaacaggaaactagcctattacccaccaatcccattccaggctgctttcaaacgcagctcaggctagaacaccagcacggggacacagctgagacttggggtttgcgacgggaacacgcccatgctgtgcctctgaatctggcaccgtcaccctgtggcctgggttcagcaacttggcctcaccttccttgtctgtgaaattcagactgggtccttgtgagatgattggagagaatgtatgaactatgtgagaacgccacctttgtgcgtatctcacgcagtgtcttccctcctttccaaagtcttctgctgtctctagacacacccgacgtggggggggggggttccctgggtctcctcctaggtctgtcccaggagggcacgcactgaaggccgcgagaatcccgggggctgcattgcgccgcgccaaggactccacacaggacctttcattttcccaactgtgctgagccaggcggccggcagagagcaggtggctgacaggccccggggagccggaccgcctgggtctaatcttcccgcagactcccttgctgtgcgctttggggcttgggcctcagtttcctcaaaaggaatgaggggcttttttggaacgttaaataatttcctacgtggttgcgggtagggagaaggagaaagagaggagcgcgcctgcgcgcctggaatcgtgcccggatcagagcaagcgctctaaaagtgttacaaacattaaggcgccaactaaaaaacccgtagtgagcgcaggcagaaaccacgggtaagagaagtggagaagcttcgcgtaggccccagggtcccgagccccgagtctcgagcgcagaatcaggggtgccaatgctctcctccgcgcccccgagcgctcgccttggccatgcgggccgccccaccgggatgagggcgctcaggccggacgctggggccccgggttctcgccccgccccgccctcggggattcagaggggccgggaggagcctcgcgcatgtgcacagctggcgccccccgccccccgcgcacagctgggacgtgggccgcggccgggcgggcgcagtcgggagccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctgtgagtgccgcgggggacgccgggggcgcggggtccggggcttcgtggagatccgggagcgcaggggtgatcggaggtggggggcgcggagggtggagggggcatcgggcgcgcggggggcctggggacttgggacgcagaagggaacctccgaagggggacgtggggggacctgggcgcggggacccgctgggcctttgttcgccctgcgggagacgccgaggggcggaacagagcgctgtgcgcgcggccttcgtagccgcctttgttcggaactcggaatccccgcaggactgggaagttgttggagcctccggggctccccccgctcgcctcccgccgccccctctcatgctccgccggcctcccgcttccccctggttcgcggcccctcctccgctcacctttcccccgctcaggacccctcggtccccctccgctccccgagcgcggcgcagccccctccgtcctcccagccccctccgccccgttcctcgtcctgttcgctcccctcctccgctcctcttcctcctccccttcctcctcctcctccccttcctcctcctcctccccttcctcctcctcctcccttcccctcctcctcccccccttccttctcctcccccagcctccgccctctccccctcccccgccccttggagcgcagtgcccaccccatccccccgcgccgg

SEQ ID NO: 11 - DNA, 2200 bp, Homo sapiens
cctcgccccctccagccggccccccgggcccctcctctcggcgcccggaccttggccctccctctcctttcccacttctctctttgccctaacttcgcccccatcccccgctcatttcctctcgcacccgggctcgccaatccctctttccaagtccctcttccagcccggccttcctctcgggttcgccccccttctccccaatctccgtcctcttccctcccttcgccctcccccccttccttcctcttcccctcacccaaccctggttcccctcgttcctcagtcccgatctctcccttactctgtccccgcccactctgcgccggcctctcagtccgggttgagccccacgtgtggacggccgcgcccccactgacagccgccgcccgccggcccgccccgcgccccgccgggcctctaaaacccccgcgccgcgccctccaccgccgcatcttctccagcgcccagcctcccgccctctctcttgctggccgcacgccccggccccgcgcacctccgcccggctccgcagccgctacccgcgcttcgttgccctgtgggactccgagcgagcccggagggaaccctcctcttcttctgggggcgacttttgtttgcttgcctgtttctttctggtgacttttgcagctttccaatatccgtcttcggagcgcacgggaatccgccgagctctgcgtgcaggcccttttttcttttgaggttcacattttttgaaattttacgccagggcttttgtaatttcctcccccgcccgctgacggtcctggagtcgctcggggctttaggccggttatgcaacgtgtaccgctcggggctgccggctgcacctccgccgcgcctcgccgctcactgcgctagacccggcgccccgcgtctcgcttcgcgggcagtcagggggccggcgctctgtcgaggtctccagctagagcagggagcccgagcccgagggagtccccggagccgacgaagggcttattagaccctgactcttttctgaggcgcgcagattttgtctttgatcactccctctccgcgggtctacggccgcgcgctttcggcgccggcgatggggagaagacggaggctgtgtctccagctctacttcctgtggctgggctgtgtggtgctctgggcgcagggcacggccggccagcctcagcctcctccgcccaagccgccccggccccagccgccgccgcaacaggttcggtccgctacagcaggctctgaaggcgggtttctagcgcccgagtatcgcgaggagggtgccgcagtggccagccgcgtccgccggcgaggacagcaggacgtgctccgagggtaagtgggcaagcggctccgcacctagggctccggcttgggggaggggggaatcctcagtttggcggctttctggcccactccgtcccagaccctttagctggagcctagagctgcagccccctttgccagaatatccaaagacccccaggagcgcgtcccccttttccttcccaaccccgcagctcagcgggcggaaagccctctctccgggggttgggcggcgggtggttagggggtccaggggtgccgatcgcagagcgtgtgcagagctcgcgctgcgggaacaggttctgaatgtccggcggcaggcgggcctgggtccgcctgctgcaggggccagagaagcctgcttgctccccacgtcggggccgccgctcgtgagccttttgtttgaggacgtgtgcagggttcacagctcaccttctcatcgtcaacccgagcgctccaccttgcgacgcgctttccttgacacgtcggggccaaagtaacagttgaccaaggaggaatggatttgggaaggagggcaaggattctttggaacggaatggtccctttgttctctgcatctggaagctagaatagtagcaaattatatgtttccatgcctcttttcgccctttaaaaaggcaggcaagggacgacagatgaaaggcagtgtttagacatttctgaccctcctgcattccagcatctagctcttttgcttccacgtctgcctcccgatctccaataatttgaagtgtaattttgatttgtttgttgtcctgaaatctactcgctcggggcattgcttacgaagaccgtttatatgttgctgcatccctctacctatctgttacgtgaccgcgcttgt

SEQ ID NO: 12 - DNA, 2000 bp, Homo sapiens
ttggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggcccagggcagaaaagtccaggttcccatgcggcctgggcccacgtggagcgggcgctgaatcaccgttcagccgcccccctcccctcctccccgaccggtgcccgcagtccccgcctcctcggccgccgcctccacggggcggggccctggcccgggaccagcgccgcggctataaatgggctgcggcgaggccggcagaacgctgtgacagccacacgccccaaggcctccaagatgagctacacgttggactcgctgggcaacccgtccgcctaccggcgggtaaccgagacccgctcgagcttcagccgcgtcagcggctccccgtccagtggcttccgctcgcagtcgtggtcccgcggctcgcccagcaccgtgtcctcctcctataagcgcagcatgctcgccccgcgcctcgcttacagctcggccatgctcagctccgccgagagcagccttgacttcagccagtcctcgtccctgctcaacggcggctccggacccggcggcgactacaagctgtcccgctccaacgagaaggagcagctgcaggggctgaacgaccgctttgccggctacatagagaaggtgcactacctggagcagcagaataaggagattgaggcggagatccaggcgctgcggcagaagcaggcctcgcacgcccagctgggcgacgcgtacgaccaggagatccgcgagctgcgcgccaccctggagatggtgaaccacgagaaggctcaggtgcagctggactcggaccacctggaggaagacatccaccggctcaaggagcgctttgaggaggaggcgcggttgcgcgacgacactgaggcggccatccgcgcgctgcgcaaagacatcgaggaggcgtcgctggtcaaggtggagctggacaagaaggtgcagtcgctgcaggatgaggtggccttcctgcggagcaaccacgaggaggaggtggccgaccttctggcccagatccaggcatcgcacatcacggtggagcgcaaagactacctgaagacagacatctcgacggcgctgaaggaaatccgctcccagctcgaaagccactcagaccagaatatgcaccaggccgaagagtggttcaaatgccgctacgccaagctcaccgaggcggccgagcagaacaaggaggccatccgctccgccaaggaagagatcgccgagtaccggcgccagctgcagtccaagagcatcgagctagagtcggtgcgcggcaccaaggagtccctggagcggcagctcagcgacatcgaggagcgccacaaccacgacctcagcagctaccaggtaggaaccgcggctgcgcggccagcctgcgccagcgccagcgccgcgcgcccccgacacttgggctcgtgcccaggcgccctctccgccgcgctccctggtggccgctcgctagagcacgcgcgccgcagacctagggtatttgcggatcagcgtcctcgcccatctcatcctccacactccgcccccacccacctgccccagctgctaagggtcttgacctttttcagaaacgtgcatcttttccagttctaattttgcacgcttgcacgtttaaagcaggagggatgaattcggtagtggataa

SEQ ID NO: 13 - DNA, 2300 bp, Homo sapiens
tcagattgtcattgggagggtgaataaatgaatgcttgcattatgagagtttgggggcagaaatatgccacagactcttatctgaagccatcagatttagtggctgcgaacccaccgaagtcagggatttacattttttacagcaacgagagaaaacttcccctttcctctgcagaagtcaggactggatctcaaaaatagaaatgtgtcctcctaaatgtgtgcccatccccgtggttgacaaacaacggatttcccaagatagctgccacacacttggtttctaatctctgtattgcttccccgccagaatgtcgaagtccttcccgaatatgcccagtcatactttctgaacttttgagcaaacaccgtccggcttcttgtgctttcctcaaagaccccaggcaccggcagggaggacacaggccggggcagagcgcccctgcgcgggggattcctgccactccgcgccagcctgcggcgcaaacgctcttctcagccgcagtcccacccgctgctggcaatctgaatgaggagccgcgctatttttacctccccggctgcaatcctttatatttacatgcaggaagcaaatatataagggattaagaaggagatgcgtggccttagtttatccagagcaggaagaggttggaataggagagggtatgtgaagtctggggtggtggaaaaggcaggtggacttcggctggttgttttctcccgatcatccctgtctctggcctggaaacccccgtactctctttcttctggcttatccgtgactgccggctccccctccaccgcccccatcttttgaggtaccacccgtcacctccgatgctgcttgggctgctgcatcactctgctgctttacccccttccccgccccccaacaaagcatgcgcagtgcgttccgggccaggcaacagcagcagcacagcatccagcaacagcatcagcacccgaagccccgctcgggcgcgctctcggggggcggggcgcacgcccgctccgcgcgtccccgcgccgctcgctcccgcgcgtccccgcgccgctcgctcccgcgcgccgcctcagcatcctcaggcccggcggcagcccccgcagtcgctgaagcggccgcgcccgccgggggagggagtagccgctggggaggctccaagttggcggagcggcgaggacccctggactcctctgcgtcccgccccgggagtggctgcgaggctaggcgagccgggaaagggggcgccgcccagccccgagccccgcgccccgtgccccgagcccggagccccctgcccgccgcggcaccatgcgcgccgagccggcgtgaccggctccgcccgcggccgccccgcagctagcccggcgctctcgccggccacacggagcggcgcccgggagctatgagccatgaagccgcccggcagcagctcgcggcagccgcccctggcgggctgcagccttgccggcgcttcctgcggcccccaacgcggccccgccggctcggtgcctgccagcgccccggcccgcacgccgccctgccgcctgcttctcgtccttctcctgctgcctccgctcgccgcctcgtcccggccccgcgcctggggggctgctgcgcccagcggtgggtatggccccgtgccctttgcgttggctttcccgcggggccctgcagaggaaagcgaagggcgcgcgggtccgtgtgctccgggcttgtccccggctcggcctttccttccctccctgcctgtctttccacccttctcgttcccaaacccccattcatcccagttcacttttggaagtccatttctgttgcattcgcgaaaaacccattccaattcttgttggttccactgggaggtgtttagtggatcctgggtccctcagcgatctctgtgcaacttgcggaggggcaaccagtggatgggaaatacagcgagggagcaagttgctacttgcgtggtggaaccttaatgtgaatgcggggaggatgtagtgataatagtggtaatgggctgtttcctcaaatttcgtatccggcgcattcagtgcggttggaattaaggtgggggaggcacacttcggggaccaaagaattaaggtgctgaagacatacttcatgcacgacctttggttctgatttctcaaagtgcttgtcattataatgaacaattaatataataccatcttctatatattgatgattggaagtcactgaaagcagaaagctggctttgtcaggaaaataaaaagaaattgggaagctgccagcatctgtatccctacatggc

SEQ ID NO: 14 - DNA, 3000 bp, Homo sapiens
tactgccgactttaggtctctctggatctcaggcccccttctctaagatgcatcctagaggaccaaaaatacactttatttgggcttcgcctgcttttgtggaagggtagtttactagaggatataatctcgtgttttaatttgctctctctcctaaaggaaatgtggagaaaaaaaaaaagcagaaattggaaataaccaatatttagtttatttcattcgattcttaggggaactggtgaggagcctaagatgattttcccttcctagagaaagaatccaaagtccagggaaatagcgacaggggagttcaagactgcccctgctagtccttccttggctactctccgctgcgatcgcaggatagctctcattagcaggagaatcgggcaagtgtgtggataagtagagagtgtgttgaacaacttgtaacgttttatgaaatacgcattgtcatggttccctaaaaggctttgcggaagccgtttgtctttactaatcaagtctttacttacacaaaagtagaagtagaagtagttttagaaaacatactaacaatcttctatccccttgaagaccagagtagcagaaaacaggtgatttgcattataaaattgcactcactttttcctcctttcagatttcacattacattagcccatttgtgttacggtgtataaaaaatggaacaggcgcctccactgcattgttctcctttaaaaatagatcacttacaccctaactttgttttccttaaattcgattcttaacaggagagctttctattatttcagatggagtgaggttgcacgactgggatggaagaaaggaatcccttaaatttgggggaatttctgttctctgttccaagaccattttacttggggtgtgggggtgggcgcggcggtcagggcagtggaacgcagtcgcggctgcgccatccctgcacttccaggcgcgcgggagggaccggcggggacgcgagctgcggactctggcgaactcgggggaggcagacagggggaggcggacacccagccggcaggcgtctcagcctccccgcagccggcgggcttttctcctgacagctccaggaaaggcagaccccttccccagccagccaggtaaggtaaagactgctgttgagcttgctgttactgagggcgcacagaccctggggagaccgaagcttgccactgcgggattctgtggggtaacctgggtctacggaagtttcctgaaagaggggagaagggtttgcatttttcctatggaggattcttctctctctagcatttcgtttgatgtattcaactggtagaagtgagatttcaacaggtagcagagagcgctcacgtggaggaggtttggggcgccgcggcgccacccccacccctcctcgggaccgcgcctatttctaaagttacacgtcgacgaactaacctatgctttaaattcctctttccagccccgtgagtccgcggcgacattgggccgtggggtggctgggaacggtcccctcctccggaaaaaccagagaacggcttggagagctgaaacgagcgtccgcgagcaggtccgtgcagaaccgggcttcaggaccgctgagctccgtagggcgtccttgggggacgccaggtcgccggctcctctgccctcgttgagatggacaacgcctcgttctcggagccctggcccgccaacgcatcgggcccggacccggcgctgagctgctccaacgcgtcgactctggcgccgctgccggcgccgctggcggtggctgtaccagttgtctacgcggtgatctgcgccgtgggtctggcgggcaactccgccgtgctgtacgtgttgctgcgggcgccccgcatgaagaccgtcaccaacctgttcatcctcaacctggccatcgccgacgagctcttcacgctggtgctgcccatcaacatcgccgacttcctgctgcggcagtggcccttcggggagctcatgtgcaagctcatcgtggctatcgaccagtacaacaccttctccagcctctacttcctcaccgtcatgagcgccgaccgctacctggtggtgttggccactgcggagtcgcgccgggtggccggccgcacctacagcgccgcgcgcgcggtgagcctggccgtgtgggggatcgtcacactcgtcgtgctgcccttcgcagtcttcgcccggctagacgacgagcagggccggcgccagtgcgtgctagtctttccgcagcccgaggccttctggtggcgcgcgagccgcctctacacgctcgtgctgggcttcgccatccccgtgtccaccatctgtgtcctctataccaccctgctgtgccggctgcatgccatgcggctggacagccacgccaaggccctggagcgcgccaagaagcgggtgaccttcctggtggtggcaatcctggcggtgtgcctcctctgctggacgccctaccacctgagcaccgtggtggcgctcaccaccgacctcccgcagacgccgctggtcatcgctatctcctacttcatcaccagcctgagctacgccaacagctgcctcaaccccttcctctacgccttcctggacgccagcttccgcaggaacctccgccagctgataacttgccgcgcggcagcctgactcccccagcgtccggctccgcaactgcccgccactcctggccagcgagggaggagccggcgccagagtgcgggaccagacaggccgcctaggcctcctggggaaaccgactcgcgccccatacccgacctagcagatcggaagcgctgcgactgtgcccgcaggttgaccttgccaagccctccaggtgatgcgcggccatgccgggtgaggagaactgaggctgagatcgccacactgagggctccctaaagccgaggtggaggaagaggagggtagaggaggagggcggtattgctgggaaccgccccctccctgccctgctccctgctgccccacccgagccct

SEQ ID NO: 15 - DNA, 3000 bp, Homo sapiens
gaatacattaaagtaggggcaacccttgagcccagacttctgccatgtgaagaccctttgaaaatcctgacaaacacaggtactgcgtaagtggtcagctaattaaagaggggaggtggagctgtcctttgtgtatccaataagtacccattatctcatttgagcatgaaaagaggccactgttattactttcaagaaggaaagtaagcaggatagctcatatttttagaaccattcctcaccaaatggaataattccggtgaaaagtgggagtgaggaagaaagaaaaaaaaaacttctaatcataatgtttgggaataagaaaggaagaagaaactcacgtcaaagccgactttctcctgcagctgtaaaataaactcttaagacccttcctgctgaaactctggagaggaaaactggagtggcgggtgggctttgcctgcagctcaactctccctcgcggcgcgggcgcggctgggttcagcacctcggaaagcgcccctcgcggcgccccgggattacgcatgctccttggggcccgccgccttggccgtgcaagtgccaccgtaactggtgagagccgctggcaacccacccggagttgacaaccgcggagagacgcagacacccactgacctccaggaagctgagcgtggtggatggaactctacgatctctttctctccaaggacggaaacctcatccaagcagtcccagaggaaacggataaaggtatttgaaagggagcgagcggccccaaatcgcacaattgagcggctgggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgctgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactgacgcaggcctcaggcccagggcgcgccgtcggggcaaggtggccttccccgggcggtaaagaggtgaaaggatgaaggagggctggggggggccccatttaagaagtaggtgggaggaggatgggcagagcatggaggaggagcctgtggataggccgaggaccttctctggagaggagatgcttcgaaatcaggtggagagaggaaattggcaaagggatagagacgagccccacgggccagacagccaacctccgctccgcaccccacagcctctccttactcttcccacgctgagtagtgtgggggcgcccagaagcgaagacaagcagcaaaaatgtagagaaattggcacggggagcggggcttagccaaatgatgcacagacaattgtgcccgtttattccagcgacttctgcggagagggcagccgtcggcacaaacactcctttgcgt

SEQ ID NO: 16 - DNA, 2200 bp, Homo sapiens
gtcccccgattccctcacccatcatataacgtgtgtatttattatgtttcccgtttcctctgtctccgccagcagaatgtaaactccatgaggtcaggaatctccgagttatgttgcgccagtgtaatccaagagcccggaacagtgcctggcacacagcgggcatatggaagaacaaatgtgtgaaggtgtgaatgaatgaataattgaaagaataaatagtagttctcagcctcacagaacacgggtcacaacctcaaatgacctgctaccctgcccataaataacagagatgcaggagtaagtgctgggctgtgacctgtcaacatgctaagccgctcaaacaaaactgcccaacagcccgctggccgcctatttgcagcactgggccctgagccgcacattcccatttcgttgataaagaaactgaccagatagtttaagtggcctgctgcggaagacagagctggtgctgcaccggtcgctgcttccccagtccttttttggcctcctttctgacgcgacgcagaccccagttctggagagtctgtcactcgctccccgtggtgggagatcagaggcctggtgtccttgggagcggcgagcggtgctcggcgcaggatagaaagggagtgcgcgcccgagtcccccagatccctgggaacccgcgccaccctcccgcccctgcccatccccggccgcgctgtcagtctccattagcgctaacaggctccagacggagcgggccgggcgctgggttaatgcaatcggcgcgttacctggggcgcaggctacattaccagcccggcccccgccaggcacggccagaaccagtcagcccgcgccctgccggccgccccgcgcctccagctcttccccggccccgcccgaacgccacacggcggagcccagccccagcccgcgccctagagcctgccaaggcgccgccggtcgggggccggcagggcgcaaggcaccagggatcccctcgccgccggacacgtgagtgcgccctgagcgcgggacagggctaggtctgcctgggaggcccgggccgagacgcgccagcagagggctagcgagtttgtagtgcagtgacgttaagtgtccgagaaggctcctgtggctgttgaagtgtcgcggacctgagctggggagggggtcggcacgctgccctcagcctcggtgagttcaatcccagccatttggggcaggcgagagtgggtgaacgaggaaaagtgctgcagggtcttcagccgcccccagagggctgtcagaagtctccaactcttgagttccggcgtgccccaacctctgtttccaaatttttccagcggacgcgcgctcttttctgggaaccctgcgtccgctcagcgcgcgctcatcccagtgtctaaggcgctcccgggtggtcttgggagttgcaagtagggaggaacggccgggtaaccacctcttttccctttatccaagcagagcctcggcgtgcccccaggaccggtaaagttcctctcgccagccgcatccatgcttctggcgcggatgaacccgcaggtgcagcccgagaacaacggggcggacacgggtccagagcagccccttcgggcgcgcaaaactgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgagcgtggggtcgggctgggaatttgaatctgggaggtccactgtctgcagcggtggctgggacaggagctggaatacacacggaagggaggcgaggagacaggggcaaatctggggcgcagaaagaactggacagggctaacgggaaaaaaaaaagattggagtcctctggaaggtcattttcccaggctctttgcagagtacctcgagctcattccagcggaagtgtcaggattgggcaccctggaagcaaaacagcagaagagtgaaatcgagtcatgaccctaaagtcatggtaggggtatggatggaaaggacagaatctggggtgccaggttgggtgggggagcctgaccttttgatggtctgctggaagggaggtggagattccaagagc

SEQ ID NO: 17 - DNA, 96 bp, Homo sapiens
cacctggaaaatcgggtcactcccacccgaatattgcgcttttcagaccggcttaagaaacggcgcaccacgagactatatcccacacctggctcg

SEQ ID NO: 18 - DNA, 526 bp, Homo sapiens
gtccgcggcgacattgggccgtggggtggctgggaacggtcccctcctccggaaaaaccagagaacggcttggagagctgaaacgagcgtccgcgagcaggtccgtgcagaaccgggcttcaggaccgctgagctccgtagggcgtccttgggggacgccaggtcgccggctcctctgccctcgttgagatggacaacgcctcgttctcggagccctggcccgccaacgcatcgggcccggacccggcgctgagctgctccaacgcgtcgactctggcgccgctgccggcgccgctggcggtggctgtaccagttgtctacgcggtgatctgcgccgtgggtctggcgggcaactccgccgtgctgtacgtgttgctgcgggcgccccgcatgaagaccgtcaccaacctgttcatcctcaacctggccatcgccgacgagctcttcacgctggtgctgcccatcaacatcgccgacttcctgctgcggcagtggcccttcggggagctcatgtgcaagctcatcg

SEQ ID NO: 19 - DNA, 18 bp, Artificial Sequence
  Designed oligonucleotide primer for PCR
gtccgcggcgacattggg

SEQ ID NO: 20 - DNA, 21 bp, Artificial Sequence
  Designed oligonucleotide primer for PCR
cgatgagcttgcacatgagct

SEQ ID NO: 21 - DNA, 80 bp, Artificial Sequence
  Designed biotinated oligonucleotide for fixation
gacaacgcctcgttctcggagccctggcccgccaacgcatcgggcccggacccggcgctgagctgctccaacgcgtcgac

SEQ ID NO: 22 - DNA, 167 bp, Homo sapiens
gacaacgcctcgttctcggagccctggcccgccaacgcatcgggcccggacccggcgctgagctgctccaacgcgtcgactctggcgccgctgccggcgccgctggcggtggctgtaccagttgtctacgcggtgatctgcgccgtgggtctggcgggcaactccgc

SEQ ID NO: 23 - DNA, 19 bp, Artificial Sequence
  Designed oligonucleotide primer for PCR
gacaacgcctcgttctcgg

SEQ ID NO: 24 - DNA, 18 bp, Artificial Sequence
  Designed oligonucleotide primer for PCR
gcggagttgcccgccaga

SEQ ID NO: 25 - DNA, 473 bp, Homo sapiens
gagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttctgagtcaaagaaaggggtgacggtcgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcag

SEQ ID NO: 26 - DNA, 21 bp, Artificial Sequence
  Designed oligonucleotide primer for PCR
gagccaagatggccgaatagg

SEQ ID NO: 27 - DNA, 23 bp, Artificial Sequence
  Designed oligonucleotide primer for PCR
ctgctttgtttacctaagcaagc

SEQ ID NO: 28 - DNA, 21 bp, Artificial Sequence
  Designed oligonucleotide primer for PCR
cacctggaaaatcgggtcact

SEQ ID NO: 29 - DNA, 20 bp, Artificial Sequence
  Designed oligonucleotide primer for PCR
cgagccaggtgtgggatata

SEQ ID NO: 30 - DNA, 27 bp, Artificial Sequence
  Designed oligonucleotide probe for Real Time PCR
cgaatattgcgcttttcagaccggctt